(19) United States
(12) Patent Application Publication
(10) Pub. No.: US 2003/0211622 A1
(43) Pub. Date: Nov. 13, 2003

(54) Methods and compositions to assess oxidative brain injury

(76) Inventor: L. Jackson Roberts II, Gallatin, TN (US)

Correspondence Address: Kenneth I. Kohn, KOHN & ASSOCIATES, PLLC, 30500 Northwestern Highway, Suite 410, Farmington Hills, MI, 48334, US

(21) Appl. No.: 10/383,704

(22) Filed: Mar. 7, 2003

Related U.S. Application Data
(63) Continuation-in-part of application No. 09/342,813, filed on Jun. 29, 1999, now Pat. No. 6,620,800.
(60) Provisional application No. 60/091,136, filed on Jun. 29, 1998.

(51) Int. Cl. G01N 33/18

(52) U.S. Cl. 436/62

(57) ABSTRACT

A method to assess oxidative stress in vivo includes the steps of measuring an amount of neuroprostanes in a biological sample before the ex vivo development of neuroprostanes in a sample, comparing the measured amount of neuroprostanes to a control and assessing oxidative stress in vivo based on this comparison. There is also provided a marker for oxidated stress by an increase of neuroprostanes in a biological sample compared to a control sample. A diagnostic tool for determining the presence of a neurodegenerative disease provides for determining an increased amount of neuroprostanes in a biological sample compared to that of a control sample.

### CROSS-REFERENCE TO RELATED APPLICATIONS

This application is a Continuation-In-Part of U.S. patent application Ser. No. 09/342,813, filed Jun. 29, 1999, which claims benefit priority of U.S. Provisional Application Serial No. 60/091,136, filed Jun. 29, 1998, and which is incorporated herein by reference.

### GOVERNMENT SUPPORT

National Institute of Health GM 42057.

### BACKGROUND OF THE INVENTION

1. Field of the Invention

This invention relates to a method of assessing oxidative stress in vivo by quantification of markers and their metabolites formed by free radical mediated oxidation.

2. Description of Related Art

Free radicals derived primarily from oxygen have been implicated in the pathophysiology of a number of human diseases, such as atherosclerosis, ischemia-reperfusion injury, inflammatory diseases, cancer and aging. A variety of methods have been developed to assess oxidative stress; however, some of these methods have limited sensitivity or specificity, while others are either too invasive or not adaptable for human investigation. Halliwell, B., et al., The Measurement Of Free Radical Reactions In Humans: Some Thoughts For Future Experimentation, FEBS Letters. 213:9-14, 1987.

Unfortunately, oxidative stress is difficult to assess in humans due to lack of reliable methods to assess oxidant stress in vivo. As one author stated, “one of the greatest needs in the field now is the availability of a non-invasive test to probe the oxidative stress status of humans.” Id.

Regional increases in oxidative damage are a feature of brain tissue obtained post mortem from patients with Alzheimer's disease (AD) (reviewed in Markesbery, W. R., 1997). However, an objective index of oxidative damage associated with AD that may be assessed during life is lacking. Such a biomarker could have an important impact on the ability to test hypotheses concerning oxidative damage in AD patients by permitting repeated evaluation to follow progression of disease and to quantify response to experimental therapeutic interventions.

Lipid peroxidation is a prominent manifestation of oxidative challenge in brain (reviewed in Markesbery, W R, 1997). Recently, it has been shown that markers of lipid peroxidation are increased in cerebrospinal fluid (CSF) of AD patients compared to control subjects (Lovell et al., 1997; Montine et al., 1997). Although these studies suggest that quantification of lipid peroxidation products in CSF may provide an intra vitam index of oxidative damage to brain, the assays employed have shortcomings, including the need for large volumes of CSF and measuring highly reactive molecules, such as 4-hydroxynonenal, that limit their interpretation or widespread application.

Previously, a series of prostaglandin F₂-like compounds, termed F₂-isoprostanes (F₂-IsoPs), were disclosed that are produced by free radical-catalyzed peroxidation of arachidonic acid independent of the cyclooxygenase enzyme (Morrow et al., 1990). Significant advantages to quantifying F₂-IsoP as an index of oxidative stress are F₂-IsoP's specificity for lipid peroxidation, F₂-IsoP's chemical stability, and the relatively small tissue volumes required for F₂-IsoP's detection.

Free radicals are generally short lived and thus, indirect methods of detection are required. Pryor, W., On The Detetion Of Lipid Hydroperoxides In Biological Samples, Free Radical Biology & Medicine, Vol. 7, pages 177-178, 1989. Standard detection methods include: electron spin resonance (directly), electron spin resonance (spin trapping), thiobarbituric acid reactive substances (TBARS), detection of malonaldehyde by direct methods (such as HPLC of malonaldehyde itself or as its derivative, dinitrophenylhydrazone), detection of other oxidation products from polyunsaturated fatty acids (such as 4-hydroxynonenal), measurement of lipid hydroperoxides, detection of volatile hydrocarbons (ethane, pentane and ethylene), detection of oxidation products from lipids other than polyunsaturated fatty acids (e.g., cholesterol), oxidation of methional, methionine, or 2-keto-4-thiomethylbutanoic acid to ethylene, oxidation of benzoic acid to carbon dioxide (often with radiolabelled carbon dioxide), oxidation of phenol benzoic acid, or aspirin to hydroxylated products, determination of decreases in antioxidant levels (e.g., decreased GSH, tocopherol, or ascorbate) or of increases in the oxidized products from antioxidants (e.g., tocopherol quinone or the ascorbyl radical), detection of oxidized DNA bases (e.g., thymine glycol, 8-hydroxydeoxyguanosine), detection of oxidized products from proteins (e.g., methionine sulfoxide from methionine) or of proteins oxidized to carbonyl-containing products that then react with hydride-reducing agents, detection of adducts of DNA bases (e.g., by enzymatic hydrolysis post-labeling using P32), and chemi-luminescence methods. Id.

Also, docosahexaenoic acid (C22:6ω3)(DHA) has been the subject of considerable interest owing to the fact that it is highly enriched in the brain, particularly in gray matter, where it comprises approximately 25-35% of the total fatty acids in aminophospholipids (Salem et al., 1986; Skinner et al., 1993). Although DHA is present in high concentrations in neurons, neurons are incapable of elongating and desaturating essential fatty acids to form DHA. Rather, DHA is synthesized primarily by astrocytes after which it is secreted and taken up by neurons (Moore et al., 1991). Although the precise function of DHA in the brain is not well understood, deficiency of DHA is associated with abnormalities in brain function (Conner et al., 1992). Applicant considered the possibility that IsoP-like compounds could be formed by free radical-induced peroxidation of DHA. Because such compounds would be two carbons longer in length than IsoPs, it would be inappropriate to term these compounds IsoPs. Since DHA is highly enriched in neurons in the brain, Applicant, therefore, proposes to term these compounds “neuroprostanes” (NPs).

It would, therefore, be useful to develop additional methods for assessing oxidative stress in vivo that are neither too invasive nor limited to animal models.

### SUMMARY OF THE INVENTION

According to the present invention, there is provided a method of assessing oxidative stress in vivo by measuring an amount of neuroprostanes in a biological sample before the ex vivo development of neuroprostanes in a sample, comparing the measured amount of neuroprostanes with a control, and assessing oxidative stress in vivo based on this comparison. There is also provided a marker for oxidative stress by detecting the increase of neuroprostanes in a biological sample compared to a control sample. A diagnostic tool for determining the presence of a neurodegenerative disease that function by detecting an increased amount of neuroprostanes in a biological sample compared to that of a control sample is also provided.

### DESCRIPTION OF THE DRAWINGS

Other advantages of the present invention will be readily appreciated as the same becomes better understood by reference to the following detailed description when considered in connection with the accompanying drawings, wherein:

FIG. 1 is a diagram showing the pathway for the formation of F₄-NPs by nonenzymatic peroxidation of DHA (A-C);

FIG. 2 is a selected ion current chromatogram obtained from the analysis of F₄-NPs generated during iron/ADP/ascorbate-induced oxidation of DHA in vitro; the series of peaks in the m/z 593 ion current chromatogram represent putative F₄-NPs, and the single peak in the m/z 573 ion current chromatogram represents the [²H₄] PGF_{2α} internal standard;

FIG. 3 is a chromatogram showing an analysis of putative F₄-NPs before and after catalytic hydrogenation, in the absence of hydrogenation, intense peaks are present in the m/z 593 ion current chromatogram representing F₄-NPs and absent are peaks of significant intensity eight atomic mass units higher at m/z 601;

following catalytic hydrogenation, intense peaks appear at m/z 601, indicating that the m/z 593 compounds have four double bonds;

FIG. 4 is a chromatogram showing the formation of a cyclic butylboronate derivative of putative F₄-NPs; The M-CH₂C₆F₅ ion for the pentafluorobenzyl ester, cyclic butylboronate, trimethylsilyl ether derivative is m/z 515; in the absence of treatment of the compounds with 1-butaneboronic acid, the peaks representing the putative F₄-NPs are present in the m/z 593 ion current chromatogram, and no peaks of significant intensity are present in the m/z 515 ion current chromatogram. However, analysis of compounds treated with 1-butaneboronic acid revealed a disappearance of the m/z 593 peaks and the appearance of intense peaks at m/z 515;

FIG. 5 is a diagram showing the predicted specific α-cleavage ions of the trimethylsiloxy substituents on the side chains of the different F₄-NP regioisomer series; the α-cleavage ions for the regioisomer series designated by asterisks were prominent ions in the mass spectrum shown in FIG. 6;

FIG. 6 is an electron ionization mass spectrum obtained of putative F₄-NPs as a methyl ester, trimethylsilyl ether derivative; an intense molecular ion is present at m/z 608, the ions designated with an “A” are common ions generated from all regioisomers; the designations (17-S), (4-S), etc. indicate ions specifically generated by compounds in the 17-series, 4-series regioisomers, etc; ions further designated with an asterisk being specific α-cleavage ions of the trimethylsiloxy substituents for the different regioisomer classes as indicated in FIG. 5;

FIG. 7 is a graph showing time-course of formation of F₄-NPs during oxidation of DHA in vitro by iron/ADP/ascorbate;

FIG. 8 is a graph showing relative amounts of F₄-NPs and F₂-IsoPs formed during co-oxidation of equal amounts of DHA and AA in vitro;

FIG. 9 is a selected ion current chromatogram obtained from the analysis for F2-IsoPs and F₄-NPs esterified in whole rat brain; the peaks in the m/z 569 ion current chromatogram represent F₂-IsoPs; the peak in the m/z 573 ion current chromatogram is [²H₄]PGF_{2α}; the peaks in the m/z 593 ion current chromatogram represent F₄-NPs; the total amounts of F2-IsoPs and F₄-NPs present were 7.9 and 6.3 ng/g brain tissue, respectively;

FIG. 10 is a selected ion current chromatogram obtained from the analysis for F₂-IsoPs and F₄-NPs esterified in lipids in 1 ml of plasma; the intense peaks present in the m/z 569 ion current chromatogram represent F₂-IsoPs; the peak in the m/z 573 ion current chromatogram representing the [²H₄]PGF_{2α}internal standard; Absent are peaks in the m/z 593 ion current chromatogram representing F₄-NPs at a level above the lower limit of detection (˜5 pg/ml);

FIG. 11 shows a selected ion current chromatogram obtained from the analysis for F₄NPs in cerebrospinal fluid from a patent with Alzheimer's disease;

FIG. 12 shows a ball and wire molecular model of phosphatidylserine containing palmitate esterified in the sn-1 position and a 13-series NP (13-F₄ₜ-NP) esterified in the sn-2 position; trailing downward on the right from the polar head group above is palmitic acid; trailing downward and then curving sharply upward on the left is the NP molecule in which the cyclopentane ring is seen at the top;

FIG. 13 shows a scatter plot of VF F₂-IsoP concentration (pg/ml) versus brain weight (gm) for 22 control subjects and AD patients with best fit regression line and 95% confidence intervals (r²=0.32, P<0.01);

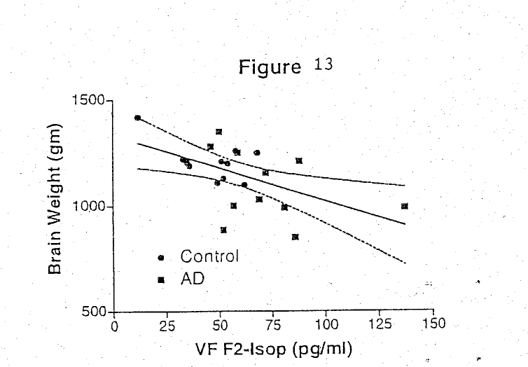

FIG. 14 shows selected ion current chromatograms from the analysis of the formation of A₂/J₂-IsoPs during oxidation of arachidonic acid in vitro; the peaks in the m/z 438 ion current chromatogram represent the syn- and anti-O-methyloxime isomers of the [²H₄]PGA₂ internal standard; in the m/z 434 chromatogram a series of peaks are present consistent with the presence of A_{2/}J₂-IsoPs; the summed total amount of the putative A_{2/}J₂-IsoPs formed being 529 ng/mg of arachidonic acid;

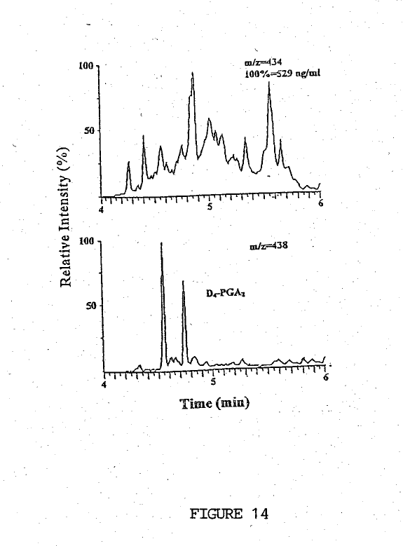

FIG. 15 is an analysis of the putative A_{2/}J₂-IsoPs formed during oxidation of arachidonic acid in vitro prior to and after catalytic hydrogenation; FIG. 1 SA, shows an analysis of compounds prior to hydrogenation; the peaks in the m/z 434 ion current chromatogram representing putative A₂/J₂-IsoPs, and the peaks in the m/z 438 chromatogram representing the [²H₄]PGA₂ internal standard; no compounds being detected six Da above m/z 434 at m/z 440 prior to hydrogenation; FIG. 15B, shows an analysis of compounds following hydrogenation; both the internal standard and the m/z 434 peaks in FIG. 15A having shifted upwards six Da following hydrogenation, indicating the presence of three double bonds;

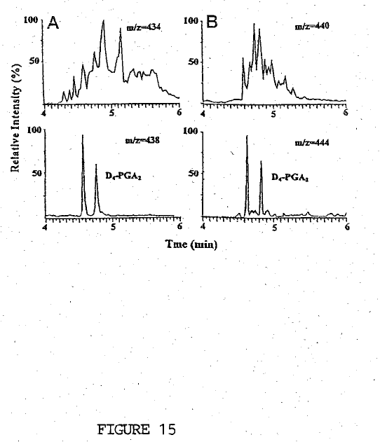

FIG. 16 is an analysis of A_{2/}J₂-IsoPs generated during oxidation of arachidonic acid in vitro as a PFB ester, piperidyl-enol-TMS ether derivative; the peaks in the m/z 566 chromatogram representing the [² H₄]PGA₂ internal standard; in the m/z 562 chromatogram being a series of peaks consistent with the formation of a piperidyl-enol-TMS ether derivative of CP-IsoPs;

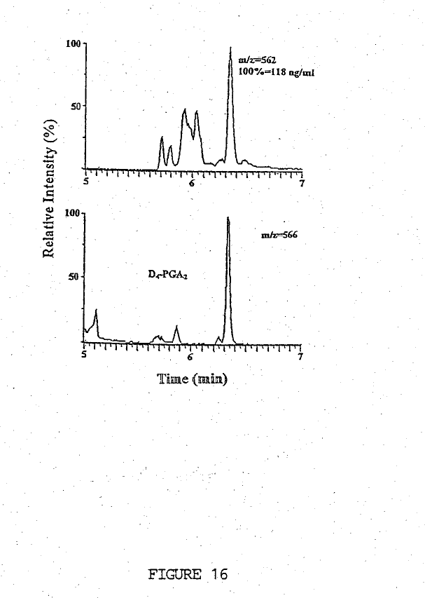

FIG. 17 shows a representative electron ionization mass spectrum obtained from the analysis of A₂/J₂-IsoPs generated from oxidation of arachidonic acid in vitro as a PFB ester, O-methyloxime, TMS ether derivative;

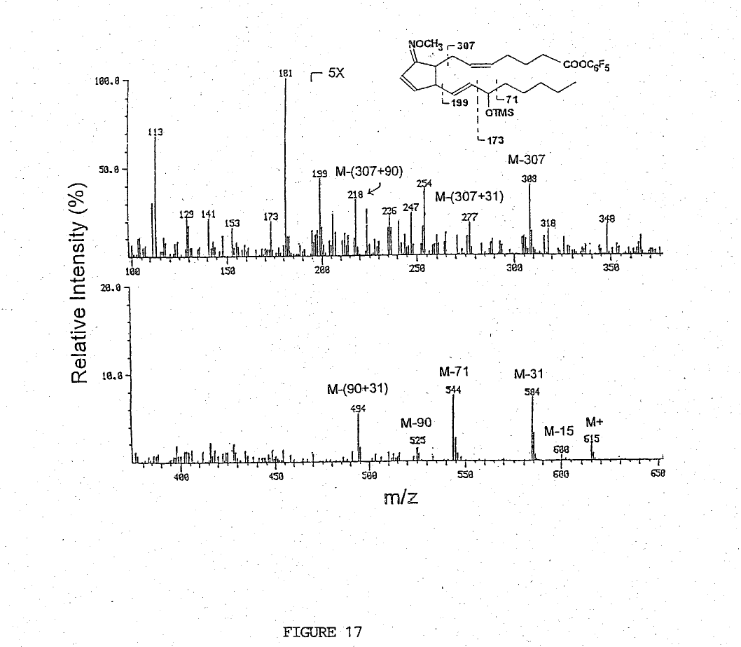

FIG. 18A is an analysis of A₂/J₂-IsoPs formed in vivo, esterified in lipids, in the liver of a rat treated with CC1₄; the peaks in the m/z 441 ion current chromatogram represent the [²H₄]PGA₂ internal standard; in the m/z 434 ion current chromatogram being a series of peaks consistent with the presence of A_{2/}J₂-IsoPs; FIG. 18B, is an analysis of A_{2/}J₂-IsoPs following oxidation of arachidonoyl-phosphatidylcholine in vitro; in the m/z 434 chromatogram is a series of peaks consistent with the presence of A₂/J₂-IsoPs in a pattern that is very similar to the pattern of peaks detected in rat liver;

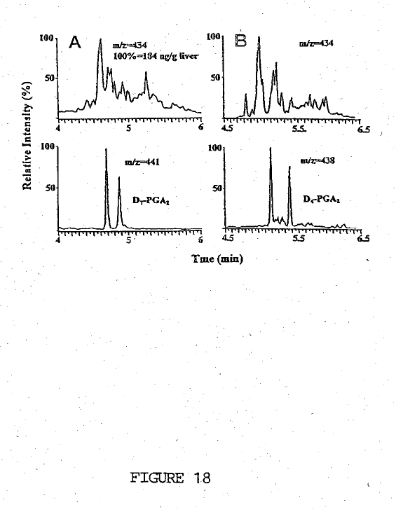

FIG. 19 shows an analysis of CP-IsoPs esterified in the liver of a rat treated with CC1₄ as a PFB ester, piperidyl-enol-TMS ether derivative, the peaks in the m/z 562 ion current chromatogram represent CP-IsoPs, and the m/z 566 peaks represent the [² H₄]PGA₂ internal standard;

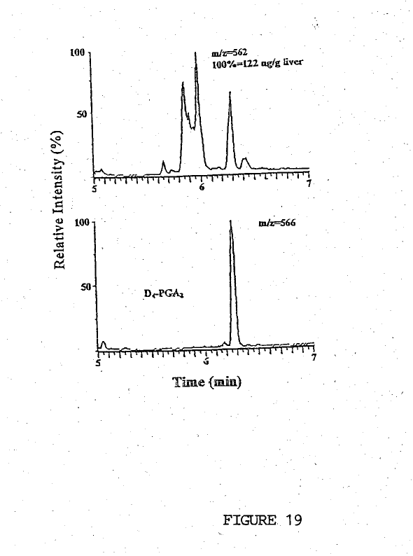

FIG. 20 shows an analysis of CP-IsoPs and D₂/E₂-IsoPs esterified in livers from normal rats and following administration of CCl₄, to induce an oxidant injury to the liver;

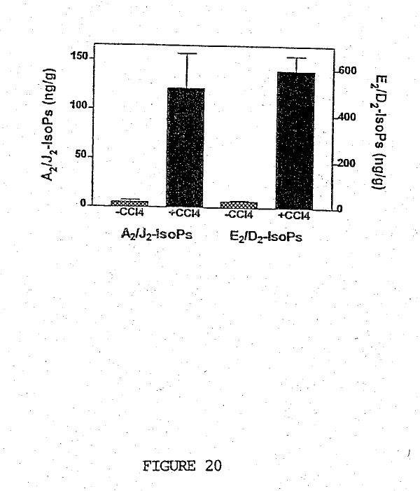

FIG. 21 shows a time course of GSH-catalyzed conjugation of 15-A₂ₜ-SOP and PGA₂ with GSH, formation of polar GSH conjugates being monitored over time and being expressed as the percent of total radioactivity that did not extract into methylene choloride;

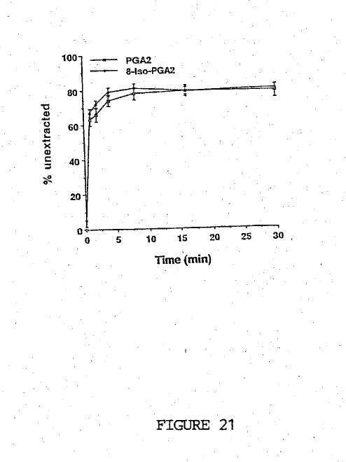

FIG. 22 shows a time course of covalent adduction of 15-A₂ₜ-IsoP and PGA₂ with albumin; formation of the adducts being monitored over time and expressed as the percent of total radioactivity present in the protein pellet following precipitation with cold ethanol;

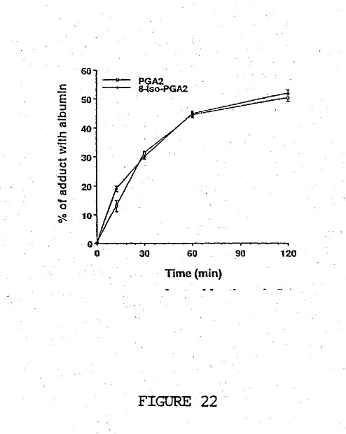

FIGS. 23A-D show liquid chromatography electrospray mass spectrometries; FIG. 23A are lactam adducts formed with lysine; FIG. 23B are hydroxylactam adducts formed with lysine; FIG. 23C are lactom adducts formed with [¹³C₆] lysine; and 23D are hydroxylactam adducts for med with [¹³C₆] lysine;

FIG. 24A and B show IsoKs FIG. 24A and NKs FIG. 24B produced by free radical oxidation of AA and DHA, respectively; FIG. 24A shows eight structural isomers of IsoKs are formed, each of which is comprised of four racemic diastereoisomers for a total of 64 compounds. The isomer shown in the figure was synthesized for use in experiments; FIG. 24B shows sixteen structural isomers of NKs are formed, each of which is comprised of eight racemic diastereoisomers for a total of 256 compounds;

FIG. 25 shows adducts produced from IsoK or NK reaction with the ε-amino group of lysine residues;

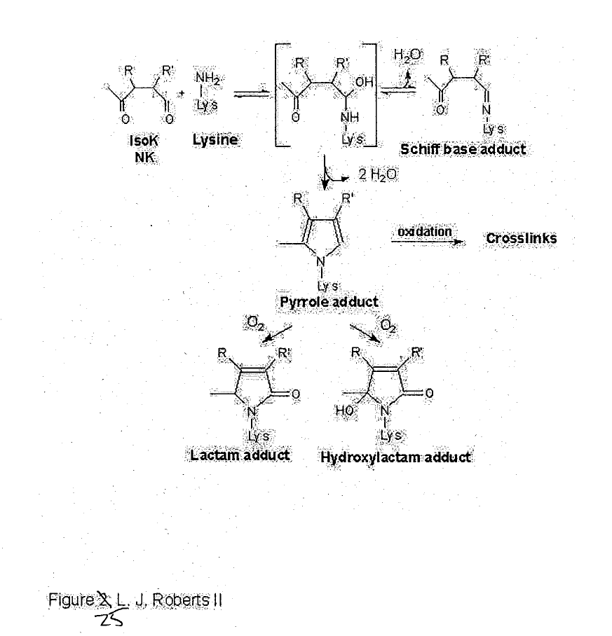

FIG. 26 shows the crosslinking of ovalbumin by IsoKs. Ovalbumin 100 μM was incubated with vehicle (lane 1), 4-HNE 1 mM (lane 2) or E₂-IsoK 1 mM (lane 3) in 1× phosphate-buffered saline pH 7.4 for 4 hours at 37° C.; after separation by SDS-PAGE (10% acrylamide), the proteins were transferred to PVDF membrane, and analyzed by Western blot with a monoclonal antibody OVA-14; the blot was developed with a chemiluminescence detection method;

FIG. 27 shows the crosslinking of Aβ by IsoKs; Aβ₁₄₂ 10 μM was incubated with vehicle (lane 1), 4-HNE 10 μM (lane 2) or E₂-IsoK 10 μM (lane 3) at room temperature in 1× phosphate-buffered saline pH 7.4 for 24 hours; the proteins were separated by SDS-PAGE on 4-12% polyacrylamide gradient gel, transferred to PVDF membrane, and analyzed by Western blot using a polyclonal antibody to Aβ₁₄₂; the blot was developed with a chemiluminescence detection method;

FIG. 28 shows the crosslinking of human tau by IsoKs; human recombinant tau protein 4 μM was incubated with vehicle (lane 1), 4-HNE 4 μM (lane 2) or E₂-IsoK 4 μM (lane 3) in 1× phosphate-buffered saline pH 7.4 at room temperature for 24 hours; the human recombinant tau protein is 65 kDa; the proteins were separated by SDS-PAGE on 4-12% polyacrylamide gradient gel, transferred to PVDF membrane, and analyzed by Western blot using anti-human tau antibody; the blot was developed with a chemiluminescence detection method;

FIG. 29 shows that the formation of the Alz50 epitope in tau is dependent on the concentration E₂-IsoKs; recombinant human microtubule associated protein-tau, 4R isoform (4 μM) was incubated with 0 to 40 μM E₂-IsoKs in 1× phosphate-buffered saline pH 7.4 at room temperature for 4 hours; dot blot was prepared by applying 6.25 μg protein for each sample directly onto a pure nitrocellulose membrane; the membrane was incubated with mouse monoclonal antibody Alz50 to paired helical filament-tau (1:100) and then incubated with a horseradish peroxidase-linked anti-mouse lgM (1:25,000; and

FIG. 30 shows tissue levels of NKs-lysyl lactam protein adducts in hippocampus and in cerebellum of AD patients and age-matched controls; values are means±SE<. Analysis of variance (ANOVA) followed by the Bonferroni test was performed to evaluate the statistical significance of difference between groups; statistical significance was assigned to the level of p<0.05; levels of NKs lactam adducts were significant for AD patients versus controls (*p<0.05) in the hippocampus and for brain region for AD (p<0.001).

### DETAILED DESCRIPTION OF THE INVENTION

Generally, the present invention provides a method of assessing oxidative stress in vivo by measuring the amount of a neuroprostane and metabolites thereof in a biological sample before the ex vivo development of neuroprostanes in a sample, then comparing the measured amount of neuroprostanes with the control sample and assessing oxidative stress in vivo based on the comparison. There is also provided a marker for oxidative stress based on the increase of neuroprostanes or metabolites thereof in a biological sample compared to that of a control sample.

Isoprostanes (IsoPs), and metabolites thereof, are prostaglandin (PG)-like compounds that are formed nonenzymatically in vivo by free radical-induced peroxidation of arachidonic acid (AA). Their formation proceeds through bicyclic endoperoxide PGH₂-like intermediates. The endoperoxide intermediates are reduced to form PGF₂-like compounds (F₂-IsoPs) (Morrow et al., 1990), or undergo rearrangement to form E-ring and D-ring compounds (E₂/D₂-IsoPs) (Morrow et al., 1994) and thromboxane-like compounds (isothromboxanes) (Morrow et al., 1996). A novel aspect of the formation of IsoPs is that, unlike cyclooxygenase-derived prostaglandins, IsoPs are formed in situ, esterified to phospholipids, and subsequently released (Morrow, et al., 1992). Quantification of F₂-IsoPs has emerged as one of the most accurate approaches to assess oxidant injury in vivo (Roberts, et al., 1997; Morrow et al., 1997; Moore et al., 1998). Furthermore, IsoPs are capable of exerting potent biological activity (Roberts et al., 1997; Morrow et al., 1997).

Cyclopentenone (CP)¹ prostaglandins (PG) of the A and J series have been shown to be produced in vitro by dehydration of the cyclopentane ring of PGE₂ and PGD₂, respectively. These compounds have attracted considerable attention because they exert unique biological actions. CP-PGs are actively incorporated into cells and accumulate in the nucleus (Narumiya et al., 1986; Narumiya et al., 1987). They have been shown to inhibit cellular proliferation with a G₁ cell cycle arrest and to induce differentiation, an effect that is related to their ability to modulate a variety of growth-related and stress-induced genes (Fukushima, 1992; Fukushima, 1990; Bui et al., 1998). These cytostatic effects can be reversible, but higher concentrations are cytotoxic and induce apoptosis (Fukushima, 1990; Kim et al., 1993; Fukushima et al., 1989). Interestingly, at very low concentrations, PGA was found to stimulate cellular proliferation (Shahabi et al., 1987). CP-PGs can also activate nuclear peroxisome proliferator-activated receptor-γ and suppress macrophage activation and inflammatory responses (Forman et al., 1995; Kliewer, 1995; Ricote et al., 1998). Furthermore, CP-PGs exhibit antiviral activity (Santoro, 1997). The common feature in these compounds is the presence of a reactive α,β-unsaturated carbonyl group, which is very susceptible to nucleophilic addition reactions and seems to be essential for many of their biological effects (Boyland et al., 1968; Atsmon et al., 1990; Honn et al., 1985).

Although the biological effects exerted by CP-PGs have been studied in some detail, the extent to which they are formed in vivo has been the subject of continuing controversy for over two decades (Attalah et al., 1974; Middledtich, 1975; Jonsson et al., 1976). Fueling this controversy has always been the uncertainty as to what extent dehydration of PGE₂ and PGD₂ ex vivo during sample processing contributes to the amount of PGA₂ and PGJ₂ detected. Recently, Δ¹²-PGJ₂ was definitely identified in human urine by Hayaishi and co-workers (Hirata et al., 1988). However, the amounts in urine from males were greater than two-fold higher than the amounts in urine from females. This is difficult to reconcile with the evidence suggesting that there is no sexual difference in the amount of PGD₂ produced in vivo in humans (Morrow et al., 1991). Convincing evidence was presented that the Δ¹²-PGJ₂ detected in urine unlikely arose as a result of dehydration of urinary PGD₂ ex vivo during sample processing. However, it is difficult to know to what extent PGD₂ undergoes dehydration in the genitourinary tract prior to voiding. This is of particular interest since the same authors recently reported that high levels of PGD synthase are present in human male reproductive organs and that seminal plasma greatly facilitates dehydration of PGD₂ (Tokugawa et al., 1998). Furthermore, the authors also recently reported that the level of PGD synthase in male urine is approximately twice that found in female urine (Melegos et al., 1996). Taken together, these findings suggest that at least some of the Δ¹²-PGJ₂ detected in urine has arisen from dehydration of PGD₂ in the genitourinary tract and explains the higher levels of Δ¹²-PGJ₂ in urine from males. This does not confute the occurrence of Δ¹²-PGJ₂ in human urine, but only raises the question of its origin, that being whether it arose from systemic sources or from local production in the genitourinary tract. Therefore, it still remains unclear whether CP-PGs are ubiquitously produced throughout the body.

Table 3 shows a comparison of the relative amounts of A₄/J₄-neuroproteins formed with that of E₄/D₄-neuroprostanes formed during oxidation of rat brain in vitro, both of which are readily detectable following oxidation of the brain.

| | Mean (ng/g tissue) | SEM |
|---|---|---|
| Normal brain E₄/D₄-NPs | 11.8 | 0.7 |
| Oxidized brain E₄/D₄-NPs | 446.3 | 81.5 |
| Normal brain A₄/J₄-NPs | 0 | — |
| Oxidized brain A₄/J₄-NPs | 98.5 | 32.6 |

Isothromboxane-like compounds are formed by oxidation of arachidonic acid (Morrow et al., J. of Bio. Chem., Vol. 271, No. 38, 1996). Accordingly, similar compounds with a thromboxane ring should be formed by oxidation of decosahexaenoic acid.

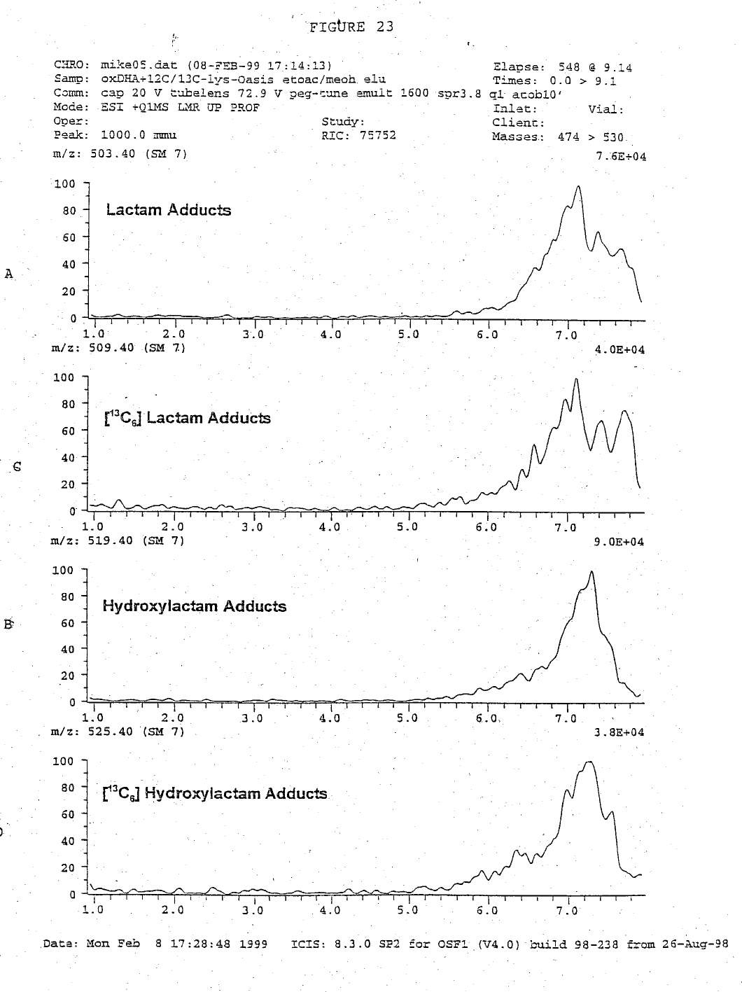

Isolevuglandin-like compounds are also produced from the oxidation of docosahexaenoic acid. These compounds readily adduct to lysine, forming lactam and hydroxylactam adducts. Experiments were performed in which docosahexaenoic acid was oxidized in the presence of a mixture of lysine and [¹³C₆] labeled lysine. This was then analyzed for lactam and hydrolactam adducts by liquid chromatography electrospray mass spectrometry. The lactam adducts formed with lysine have a mass to charge ratio of 503 and the hydroxylactam adducts have a mass to charge ratio of 519. The respective lactam and hydroxylactam adducts formed with [¹³C₆] lysine have mass to charge ratios of 509 and 525, respectively, as can be seen in FIG. 23.

Of particular interest is that IsoP-like compounds can be formed from DHA, which derives from the fact that a role for free radicals in the pathogenesis of a number of neuordegenerative diseases, e.g. Alzheimer's disease, Parkinson's disease, Huntington's disease, and amyotrophic lateral sclerosis, has been suggested (Simonian et al., 1996; Knight, 1997; Markesbery, 1997). Thus, quantification of such compounds provides a unique marker and diagnostic tool of oxidative injury in the brain. Furthermore, these compounds, like IsoPs, exert biological activity. This is supported by the finding that PGF_{4α}, the four series F-prostaglandin corresponding to the structure expected from cyclooxygenase action on C22:6, is approximately equipotent with cyclooxygenase-derived PGF_{2α}, in contracting gerbil colonic smooth muscle strips (Markesbery, 1997). In addition, the formation of NPs esterified in lipids has significant effects on the biophysical properties of neuronal membranes, which impairs normal neuronal function. This is particularly relevant, since it has been suggested that one of the physiological functions of DHA is to maintain a certain state of membrane fluidity and promote interactions with membrane proteins that are optimum for neuronal function (Salem, 1995; Dratz, 1986).

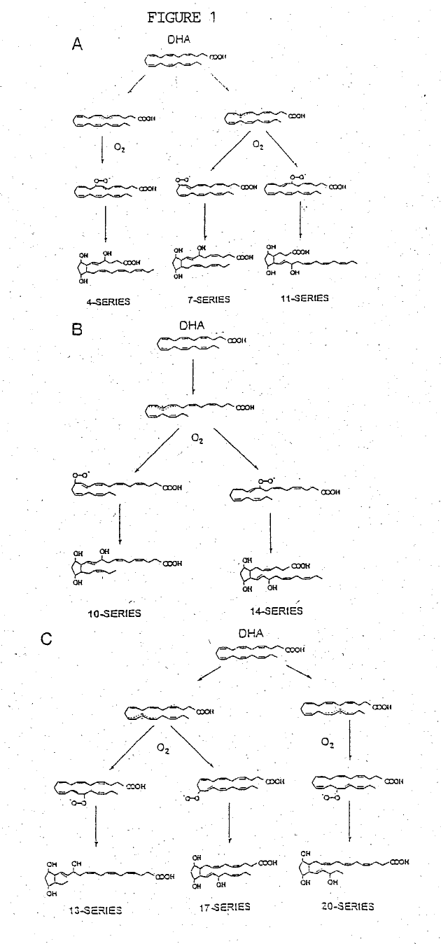

The mechanism by which F₄-NPs could be formed is outlined in FIGS. 1, A-C. As noted, five DHA radicals are initially generated, which following addition of molecular oxygen, results in the formation of eight peroxyl radicals. These peroxyl radicals then undergo endocyclization followed by further addition of molecular oxygen to form eight bicyclic endoperoxide intermediate regioisomers, which are then reduced to form eight F-ring NP regioisomers. Each regioisomer is theoretically comprised of eight racemic diastereomers for a total of 128 compounds. A nomenclature system for the IsoPs has been established and approved by the Eicosanoid Nomenclature Committee in which the different regioisomer classes are designated by the carbon number on which the side chain hydroxyl is located with the carboxyl carbon designated as C-1 (Attalah et al., 1974). Thus, in accordance with this nomenclature system, the F-ring NP regioisomers are similarly designated as 4-series F₄-NPs, 7-series F₄-NPs, etc.

Compounds were analyzed employing gas chromatography (GC) mass spectrometry (MS). Levels of putative D₄E₄-NPs increased dramatically 380-fold after oxidation from 15.2±6.3 ng/mg DHA to 5773±1024 ng/mg DHA (n=3). Subsequently, a variety of chemical methods and liquid chromatography tandem MS definitely identified these compounds as D₄E₄-NPs. The formation of D₄E₄-NPs was explored from a biological source, rat brain synaptosomes. Basal levels of D₄E₄-NPs were 3.78±0.6 ng/mg protein and increased 54-fold after oxidation (n=4). These compounds were detected in fresh brain tissue from rats at a level of 12.1±2.4 ng/g brain tissue (n=3). Thus, these studies have identified novel D/E-ring IsoP-like compounds derived from DHA. They are readily detectable in brain tissue in vivo suggesting that ongoing oxidative stress is present in the central nervous system of normal animals, and presumably humans. Identification of these compounds provides a rationale to examine their role in neurological disorders associated with oxidant stress.

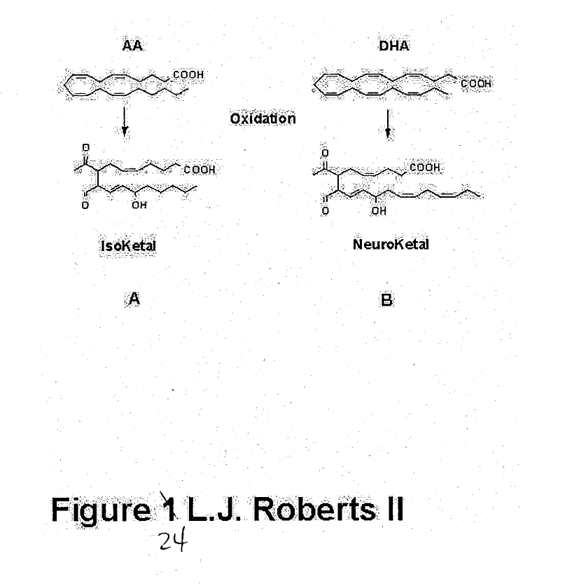

Additionally, these neuroprostanes, which are formed by the oxidation of docosehexaenoic acid are susceptible to further oxidation. This susceptibility results from the additional 1,4 diene double bonds on the side chains of the compounds. Examples of some products of the further oxidation are shown in FIG. 24. For example, the further oxidation of 4-series F₄-neuroprostane can be a single oxidation product (FIG. 24, left) or a cyclic compound (FIG. 24, right). While the F-ring compound is the only compound depicted in FIG. 24, it is important to note that the D-, E-, A-, and J-ring structures, thromboxane ring structure, and the isolevuglandins-like structure will all react in the same manner, and the side chain ring structure can also be comprised of any of the ring structures.

Also provided by the present invention are metabolites of all the neuroprostanes and isothromboxane-like compounds. These compounds are metabolized by processes of beta oxidation, omega oxidation, double bond reduction, dehydrogenation of the side chain hydroxyl groups, and in the case of E₄/D₄-and A₄/J₄-neuroprostanes, reduction of the ring carbonyl to a hydroxyl group. It was also discovered that polar glutathione conjugates and their derivatives of A₂/J₂-isoprostanes also occur and thus such conjugates can be formed from A₄/J₄-neuroprostanes.

Reactive aldehydes derived from lipid peroxidation are thought to be key effectors of oxidative injury because of their capacity to covalently modify proteins and DNA and they have been suggested to play a key role in the pathogenesis of neurodegenerative processes. 4-hydroxynonenal (4-HNE) has been extensively studied and is considered to be one of the most reactive aldehyde products of lipid peroxidation. Recent studies have shown that 4-HNE is generated following exposure of neurons to Aβ and is involved in the disruption of ion homeostasis and neuronal cell death. 4-HNE binds to the astrocytic transporter GLT-1 and the neuronal glucose transporter GLUT-3 and impairs their functions. Moreover, 4-HNE can induce crosslinking of several cytoskeletal proteins including tau and also Aβ₁₋₄₂.

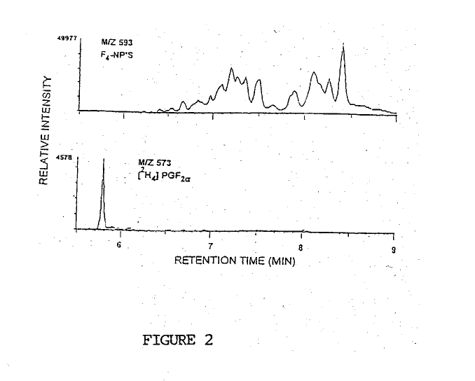

While 4-HNE is the most studied of the aldehydes derived from lipid peroxidation, the formation of a new class of highly reactive γ-ketoaldehydes that adduct proteins at a rate that exceeds that of 4-HNE by more than an order of magnitude and exhibit a unique proclivity to cross-link proteins was reported. These compounds are termed isoketals (IsoKs) and neuroketals (NKs), which are formed by the IsoP pathway and the NP pathway, respectively (FIG. 1). Sixty-four regio- and stereo-IsoK isomers are formed as products of the IsoP pathway and 256 regio- and stereo-NK isomers are formed as products of the NP pathway. IsoKs and NKs rapidly adduct to lysine residues in a time frame of a few minutes. IsoKs and NKs form an initial reversible Schiff base adduct with the ε-amine of lysine residues, which then proceeds through an irreversible pyrrole that undergoes autoxidation to yield stable lactam and hydroxylactam adducts (FIG. 2).

NKs are an attractive candidate for participating in the formation of protein aggregation/cross-linking and inducing neurodegeneration in AD for a number of reasons. Firstly, NKs are much more reactive compared to 4-HNE. Secondly, NK protein adducts are present at detectable levels in normal human brain. Thirdly, products of the NP pathway are increased in AD brain to a greater extent than products of the IsoP pathway. Finally, IsoK-adducted proteins are poorly degraded by the proteasome and they also inhibit the proteasome from degrading normal substrates, effects that would lead to protein accumulation and aggregation. Moreover, they induce cytotoxicity in P19 neuroglial cells in culture at submicromolar concentrations, which are approximately 100-fold lower than concentrations of 4-HNE required to induce cytotoxicity. Although these latter experiments were carried out using a synthetic IsoK because of the unavailability of a synthetic NK, similar effects are expected with NKs and NK-adducted proteins. Collectively, this lead to exploration of the ability of these γ-ketoaldehydes to covalently modify and crosslink Aβ and tau and whether levels of NK protein adducts are increased in the brain from patients with AD compared to aged-matched controls.

The above discussion provides a factual basis for the use of neuroprostanes as diagnostic tools for assessing oxidative stress. The methods used with and the utility of the present invention can be shown by the following non-limiting examples and accompanying figures.

### EXAMPLES

### Example 1

General Methods

Experimental Procedures

Materials

Docosahexaenoic acid, pentafluorobenzyl bromide, and disopropylethylamine were purchased from Sigma; dimethylformamide, undecane, and 1-butaneboronic acid from Aldrich; N,O-bis(tri-methylsilyl) trifluoroacetamide from Supelco (Bellefone, Pa.); [² H₉9 N,O-bis(trimethysilyl) trifluoroacetamide from Regis Chemical (Morton Grove, Ill.); organic solvents from Baxter Healthcare (Burdick and Jackson Brand, McGaw Park, Ill.); C-18 Sep-Paks from Waters Associates (Milford, Mass.); 60ALK6D TLC plates from Whatman (Maidstone, UK); and [²H₄]PGF_{2α} from Cayman Chemical (Ann Arbor, Mich.).

Oxidation of DHA

DHA and AA were oxidized in vitro using iron/ADP/ascorbate as described (Longmire et al., 1994).

Purification and Analysis of F₄-NPs

Free and esterified F₄ were extracted using a C-18 Sep-Pak cartridge, converted to a pentafluorobenzyl ester, purified by TLC, converted to a trimethylsilyl ether derivative, and quantified by stable isotope dilution negative ion chemical ionization gas chromatography mass spectrometry using [² H₄]PGF_{2α} as an internal standard using a modification of the method described for the quantification of F₂-IsoPs (Morrow et al., 1994). Instead of scraping 1 cm below to 1 cm above where PGF_{2α} methyl ester migrates on TLC for analysis of F₂-IsoPs, the area scraped was extended to 3 cm above where PGF_{2α} methyl ester migrates. This extended area of the TLC plate was determined to contain F₄-NPs by analyzing small 5-mm cuts using approaches for their identification described below. The M-CH₂C₆F₅ ions were monitored for quantification (m/z 593 for F₄-NPs and m/z 573 for [²H₄]PGF_{2α}). Quantification of the total amount of F₄-NPs and F₂-IsoPs was determined by integrating peak areas. Formation of cyclic boronate derivatives and hydrogenation were performed as described (Morrow et al., 1990). Electron ionization mass spectra were obtained using a Finnigan Incos 50B quadropole instrument as described (Morrow et al., 1994).

Analysis of F₄-NPs in Human Cerebrospinal Fluid

Cerebrospinal fluid was obtained from seven subjects following informed consent. Subjects with Alzheimer's disease (n=4) had been diagnosed with probable Alzheimer's disease during life. Control subjects (n=3) were age-matched individuals without clinical evidence of dementia or other neurological disease; each had annual neuropsychological testing with all test scores within the normal range. Ventricular cerebrospinal fluid was collected as part of a rapid autopsy protocol. Mean post-mortem intervals were 2.9±0.3 hours in control subjects and 2.7±0.2 hours in Alzheimer's patients. Brains were evaluated using standard criteria for Alzheimer's disease (Khachaturian 1985; Mirra et al., 1991). Patients with brainstem or cortical Lewy body formation, or significant cerebrovascular disease, were excluded. Control subjects demonstrated only age-associated alterations. Statistical analysis of data was performed using the unpaired t test.

Molecular Modeling of NP-containing Phosphatidylserine

Molecular modeling was performed with Macspartan computer software.

Results

A representative selected ion current chromatogram obtained from the analysis. for F₄-NPs following oxidation of DHA in vitro with iron/ADP/ascorbate is shown in FIG. 2. A series of m/z 593 peaks eluted over approximately a 90 second period beginning approximately 30 seconds after the elution of the [²H₄]PGF_{2α} internal standard. F₄-NPs would be expected to have a longer GC retention time than PGF_{2α} because their C-value is two units higher. The time scales of some of the chromatograms obtained from the analysis of F₄-NPs shown in subsequent figures are compressed or expanded compared with that in FIG. 2; this gives the impression that the relative abundances/pattern of the different isomers detected differs. Furthermore, the retention times over which the F₄-NPs elute differs somewhat in the different figures, because these analyses were performed on different days using different columns that vary somewhat in length.

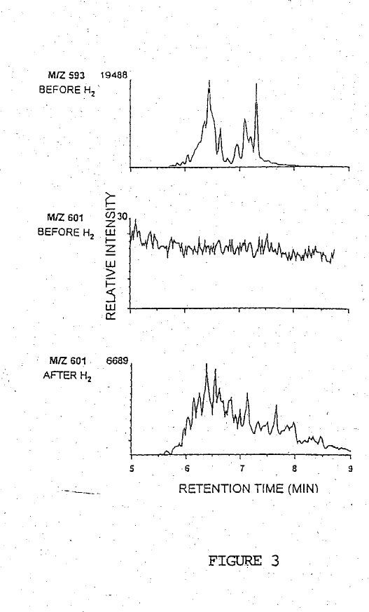

Analysis of these compounds as a [²H₉]trimethylsilyl ether derivative resulted in a shift in the m/z 593 peaks to m/z 620, indicating the presence of three hydroxyl groups. Analysis following catalytic hydrogenation is shown in FIG. 3. Prior to hydrogenation, no peaks were present eight Da above m/z 593 at m/z 601. However, following hydrogenation, intense peaks appear at m/z 601, indicating the presence of four double bonds. The pattern of the hydrogenated compounds differs significantly from that of the nonhydrogenated compounds, because hydrogenation converts the compounds into new compounds that are resolved differently than the nonhydrogenated compounds.

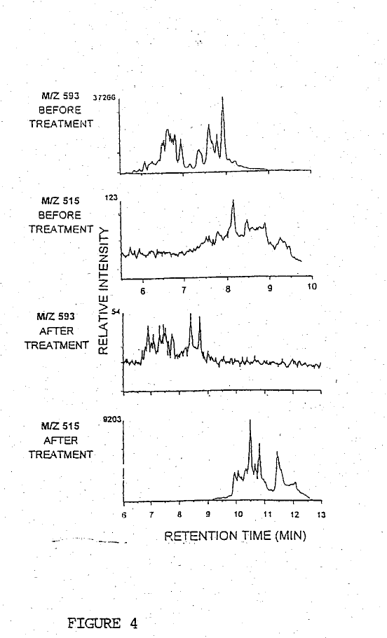

F₄-NPs are formed by reduction of endoperoxide intermediates (FIG. 1). Thus, the cyclopentane ring hydroxyls must be oriented cis, but they can be either α,α or β,β. Evidence that these compounds contained a cyclopentane (prostane) ring with cis-oriented hydroxyls was obtained by analyzing the compounds as a cyclic boronate derivative (FIG. 4). PGF₂ compounds with cis-oriented prostane ring hydroxyls form a cyclic boronate derivative bridging the ring hydroxyls (Pace-Asiak et al., 1971). The M-CH₂C₆F₅ ion for the cyclic boronate derivative is m/z 515. When the compounds were analyzed as a pentafluorobenzyl ester, trimethysilyl ether derivative, no intense peaks were present at m/z 515. However, when the pentafluorobenzyl ester derivatives were treated with 1-butaneboronic acid and then converted to a trimethylsilyl ether derivative, the intense peaks at m/z 593 were no longer present and intense peaks appeared at m/z 515. Again, the pattern of the m/z 515 peaks differs from that of compounds that were not treated with 1-butaneboronic acid because of differences in resolution of the individual compounds as a cyclic boronate derivative.

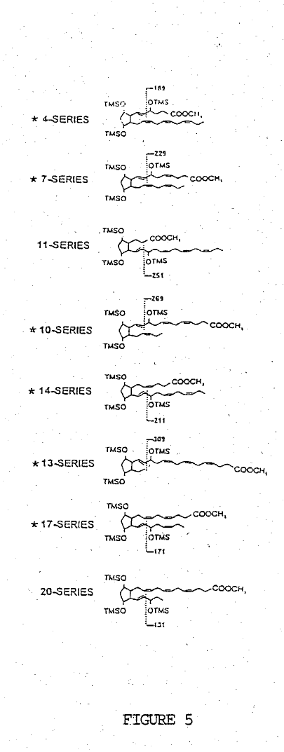

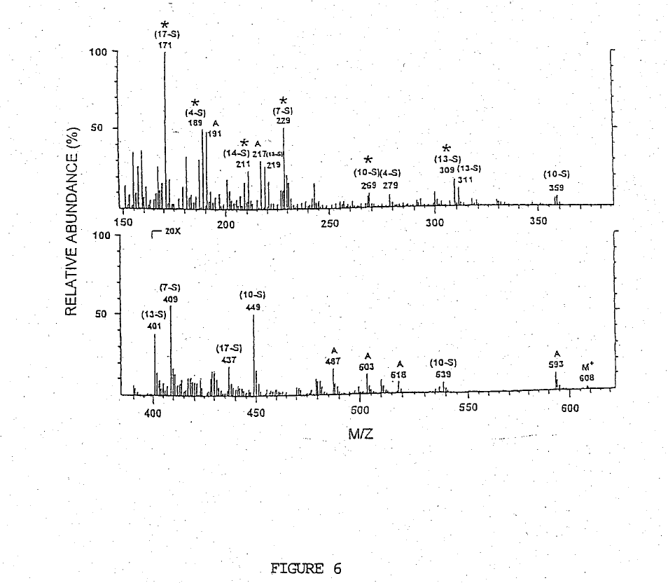

Finally, these compounds were subjected to analysis by electron ionization mass spectrometry as a methyl ester, trimethylsilyl ether derivative. Multiple mass spectra consistent with compounds representing the different regioisomers of F₄-NPs eluted from the GC column over approximately 45 seconds. This elution time differs from that of the pentafluorobenzyl ester derivatives used for negative ion chemical ionization, because methyl esters elute from the GC column much earlier and thus the duration over which they elute is compressed. When analyzed by electron impact mass spectrometry, the different F₄-NP regioisomers are expected to give characteristic α-cleavage ions of the trimethylsiloxy substituents on the side chains (FIG. 5). One of the mass spectra obtained is shown in FIG. 6. The ions designated with “A” are ions that are generated from all of the different regioisomers. These include, in addition to the molecular ion at m/z 608, m/z 593 (M-15, loss of CH₃) m/z 539 (M-90, loss of Me₃SiOH), m/z 518 (M-2×90)), m/z 501 (M-(90+15)), m/z 487 (M-121, loss of OCH₃+90), m/z 217 (Me₃SiO—CH═CH═O⁺SiMe₃), a characteristic ion of F-ring prostanoids (Pace-Acsiak, 1989), and m/z 191 (Me₃SiO⁺═CH—OSiMe₃), a rearrangement ion characteristic of F-ring prostanoids (Pace-Asciak, 1989). The ions designated (17-S), (4-S), etc. indicate ions generated specifically from 17-series, 4-series, etc. regioisomers. These include the following: (a) 10-series regioisomer ions m/z 539, (M-69, loss CH₂(CH₂)₂CH₃), m/z 449 (M-(69+2×90)), (b) 17-series regioisomer ion m/z 437 (M-171, loss of CH(Me₃SiOH)CH₂CH═CHCH₂CH₃), (c) 7-series regioisomer ion m/z 409 (M-(109+90), loss of CH₂CH═CHCH₂CH═CHCH₂CH₃+90), (d) 13-series regioisomer ions m/z 401 (M-207, loss of CH₂CH═CHCH₂CH═CHCH₂CH═CH(CH₂)₂COOCH₃), m/z 311, (M-(207+90)), m/z 219 (M-(309+90), loss of CH(Me₃SiOH)CH₂CH═CHCH₂CH═CHCH₂CH═CH(CH₂)₂CO OCH₃+90), and (e) 4-series regioisomer ion m/z 279 [M-(149+2×90), loss of CH₂CH═CHCH₂CH═CHCH₂CH═CHCH₂CH₃+2×90). The six ions further designated with an asterisk represent specific α-cleavage ions of the trimethylsiloxy substituents of different regioiosomers as shown in FIG. 5. These data indicated that this was a mass spectrum of a mixture of six of the eight regioiosomers co-eluting simultaneously from the GC column. This evidence for the presence of predicted six out of eight regioiosomers supports the proposed mechanism of formation of these compounds outlined in FIG. 1.

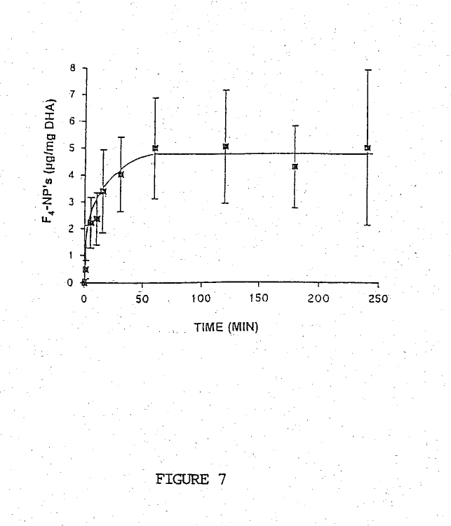

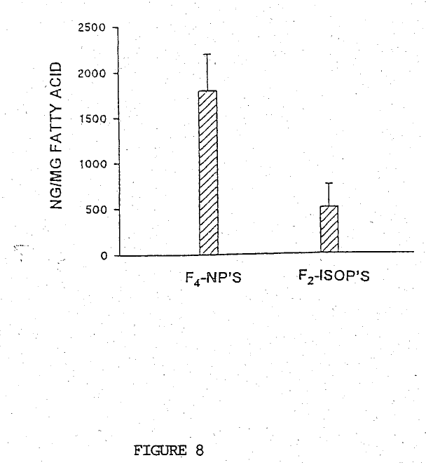

The time course of formation of F₄-NPs during oxidation of DHA using iron/ADP/ascorbate was rapid, reaching a maximum level of approximately 5 μg/mg DHA at 50 minutes (FIG. 7). The amounts of F₂-IsoPs formed from oxidation of AA were compared with the amounts of F₄-NPs formed from DHA. In these experiments, equal molar amounts of AA and DHA were co-oxidized with Fe/ADP/ascorbate and the total amounts of F₂-IsoPs and F₄-NPs generated quantified. Interestingly, the relative amounts of F₄-NPs formed exceeded that of F₂-IsoPs by a mean of 3.4-fold (FIG. 8).

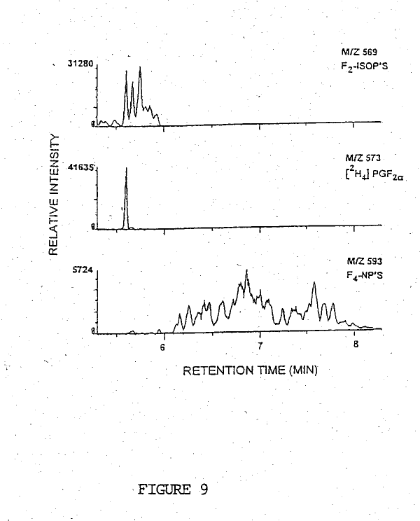

Experiments were undertaken to determine whether F₄-NPs are present esterified in brain lipids in vivo (Table I). Both F₂-IsoPs and F₄-NPs were present at readily detectable levels esterified in lipids of normal whole rat brain at levels of 10.3±3.1 and 7.0±1.4 ng/g, respectively (n=4). A selected ion current chromatogram obtained from one of these analyses is shown in FIG. 9. Although the levels of F₂-IsoPs were slightly higher than the levels of F₄-NPs, these differences were not significant (p>0.05). However, levels of F₄-NPs esterified in the cortex of newborn pig brain (13.1±0.8 ng/g) greatly exceeded levels of F₂-IsoPs (2.9±0.4 ng/g) by a mean of 4.5-fold (n=3) (p<0.0001). Note that the pattern of F₄-NP peaks detected esterified in brain differs somewhat than that of compounds formed by oxidation of DHA in vitro. Slight differences were observed in the pattern of F₂-IsoPs formed from oxidation of arachidonic acid in vitro compared with that of compounds present esterified in tissue lipids. Although the reason for these differences has not been firmly established, a reasonable explanation for this is that there are steric influences of phospholipids on the formation of different isomers from esterified substrate.

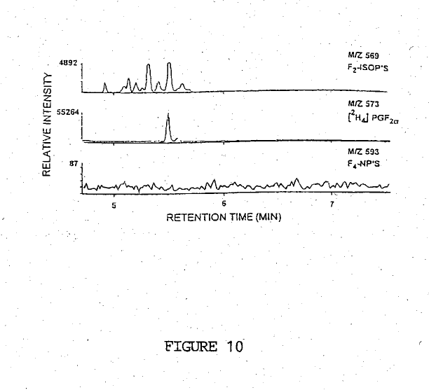

As a measure of specificity of the assay to detect esterified F₄-NPs in tissues,F₄-NPs esterified were analyzed for lipids in 1 ml of human plasma, which contains only very small amounts of DHA (FIG. 10) (Salem et al., 1986). Intense peaks were present in the m/z 569 ion current chromatogram representing F₂-IsoPs, but absent were peaks of significant intensity in the m/z 593 ion current chromatogram that indicate the presence of F₄-NPs at levels above the lower limits of detection (˜5 pg/ml).

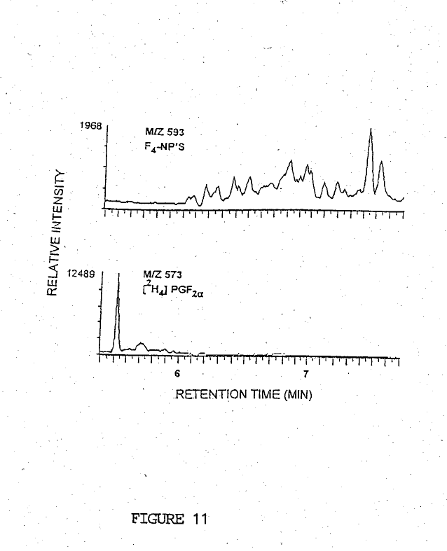

Although F₄-NPs can be readily detected esterified in the brain, the utility of such measurements to assess oxidative injury is restricted to animal models of neurological disorders or brain samples obtained post-mortem from humans. It was therefore examined whether F₄-NPs could be detected in cereobrospinal fluid obtained from four patients with Alzheimer's disease and three age-matched control subjects. F₄-NPs were detected in 1-2 ml of cerebrospinal fluid from the control subjects at a level of 64±8 pg/ml. Of considerable interest was the finding that the concentrations measured in the patients with Alzheimer's disease were significantly higher (110±12 pg/ml) (p<0.05). A selected ion current chromatogram obtained from the analysis of F₄-NPs in cerebrospinal fluid from a patient with Alzheimer's disease is shown in FIG. 11. The pattern of F₄-NP peaks detected in free-form in cerebrospinal fluid differs somewhat from the pattern peaks detected esterified in tissue phospholipids (FIG. 9). Similar differences have been observed for the pattern of F₂-IsoP peaks detected in free form in plasma and urine compared with the pattern of peaks detected esterified in tissue phospholipids as free compounds following base hydrolysis of a tissue lipid extract. Although the reason for these differences has not been established, this is explained by differences in the efficacy of phospholipases to hydrolyze different isomers from phospholipids. Cerebrospinal fluid concentrations of F₂-IsoPs were similarly increased in patients with Alzheimer's disease but were lower than the levels of F₄-NPs in both control subjects and Alzheimer's patients (46±4 and 72±7 pg/ml, respectively).

Discussion

These studies have elucidated a new class of F₂-IsoP-like compounds formed in vivo by free radical-induced peroxidation of DHA. Free radical-induced peroxidation of AA results not only in the formation of F-ring IsoPs but also D-ring and E-ring IsoPs and thromboxane-like compounds (isothromboxanes) (Morrow et al., 1994; Morrow et al., 1996).

One of the motivations for determining whether IsoP-like compounds could be formed as peroxidation products of DHA involves the possibility that quantification of these compounds provides a unique marker of oxidative injury in the brain that can be exploited to investigate the role of free radicals in the pathogenesis of neurological disease.

Although invasive, cerebrospinal fluid is frequently obtained for diagnostic purposes in patients with suspected neurological disorders. Thus, the availability of a marker of oxidative injury in the brain that can be measured in cerebrospinal fluid intra vitam is an important advance. Thus, the finding that F₄-NPs can be detected in human cerebrospinal fluid clearly has important clinical applications. It was shown that markers of lipid peroxidation are increased in cerebrospinal fluid of patients with Alzheimer's disease (Lovell et al., 1997; Montine et al., 1997). However, these assays have shortcomings related to measurement of reactive molecules, i.e., 4-hydroxynonenal, and require large volumes of fluid. However, F₄-NPs were detectable using negative ion chemical ionization mass spectrometry in 1-2 ml of cerebrospinal fluid from normal subjects, an amount that can usually be obtained safely from patients for diagnostic purposes. Although it was a limited study, the finding that F₄-NP concentrations in cerebrospinal fluid from patients with Alzheimer's disease were significantly higher than levels in age-matched control subjects highlights the potential of this approach to provide insights into the role of free radicals in the pathogenesis of neurological disorders. Another important aspect of this finding is that serial measurements of F₄-NPs in cerebrospinal fluid might provide a biochemical assessment of disease progression as well as a means to monitor efficacy of therapeutic intervention, e.g., with antioxidants, during life. No other method has proven to be reliable to obtain such information.

One question that arises is whether there is a distinct advantage of measuring either IsoPs or NPs to assess oxidative injury in the brain. It is of interest that the relative amounts of F₄-NPs formed during oxidation of DHA in vitro exceeded the amounts of F₂IsoPs generated from an equivalent amount of AA by as much as 3.4-fold (FIG. 8). This is consistent with the fact that of the naturally occurring fatty acids, DHA is the most easily oxidizable (Dratz et al., 1986). This suggests that measurement of F₄-NPs in some situations provides a more sensitive index of oxidative injury in the brain than measurement of F₂IsoPs. The ratio of levels of AA and DHA, and thus the capacity to form IsoPs and NPs, respectively, varies significantly between different regions of the brain (white matter, gray matter), different cell types (neurons, astrocytes, oligodendrocytes), and subcellular fractions (myelin, synaptosomes) (Salem et al., 1986; Skinner et al., 1993; Bourre et al., 1984). In this regard, it was found that levels of F₄-NPs and F₂IsoPs esterified in whole rat brain were similar, whereas levels of F₄-NPs were higher than levels of F₂IsoPs in the cortex of newborn pigs and in human cerebrospinal fluid. Therefore, there are distinct advantages associated with measuring either IsoPs or NPs to assess oxidant injury in the brain depending on the site of oxidant injury and the predominant cell types involved. Thus, the best approach at this time is to quantify both IsoPs and NPs in a variety of situations involving different types of oxidative insults to the brain both in experimental animals and in human neurological disorders. The practicality of this approach is facilitated by the fact that the method of assay developed allows simultaneous measurement of both F₄-NPs and F₂IsoPs in the same sample.

There are additional ramifications that are relevant to neuropathobiology that emerge from this discovery. Two of the IsoPs, previously referred to as 8-iso-PGF_{2α} and 8-iso-PGE₂, now termed 15-F₂ₜ-IsoP and 15-E2t-IsoP according to the approved nomenclature for IsoPs (Taber et al., 1997), have been found to possess potent biological activity ranging from effects on vascular and bronchial smooth muscle, endothelin release, platelet function, to cellular proliferation (Roberts et al., 1997; Morrow et al., 1997). Of interest has been the evidence obtained which suggests that these IsoPs exert their vascular effects by interacting with a unique receptor (Roberts et al., 1997; Morrow et al., 1997). Thus, NPs also are found to possess important biological actions that are relevant to the pathophysiology of oxidant injury to the brain. As mentioned, this is greatly supported by the finding that C22-PGF_{4α} is bioactive (Salem et al., 1986). This compound is one of the F₄-NPs that is formed, although, analogous to IsoPs, compounds in which the side chains are oriented cis likely predominate over compounds in which the side chains are oriented trans in relation to the cyclopentane ring (Morrow et al., 1990). However, in the case of the IsoPs, inversion of the stereochemistry of the upper side chain of PGF_{2α} and PGE₂ affords different and/or more potent biological actions (Roberts et al., 1997; Morrow et al., 1997).

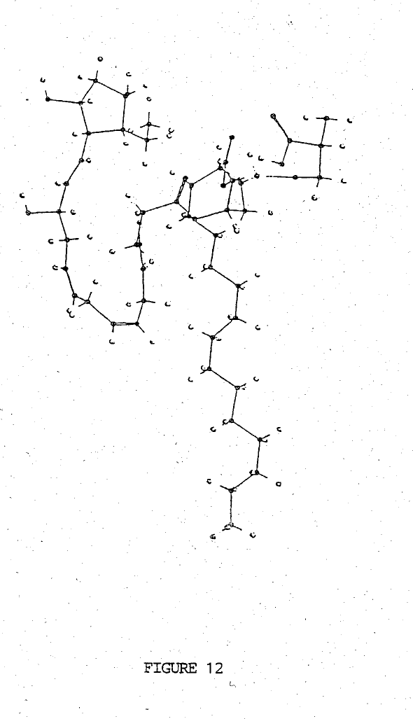

In addition, phospholipids containing esterified NPs are very unnatural and unusual molecules. Shown in FIG. 12 is a molecular model of phosphatidylserine with palmitate esterified at the sn-1 position and a 13-series NP (13-F₄ₜ-NP) esterified at the sn-2 position. Mass spectral evidence for the formation of 13-series F₄-NPs during oxidation of DHA was presented in FIG. 6. Trailing downward on the right from the polar head group above is palmitic acid. Trailing downward and then curving sharply upward on the left is the NP molecule in which the cyclopentane ring is seen at the top. Unmistakably, this is a remarkably distorted molecule. Thus, enhanced formation of these unusual phospholipids in neuronal membranes in settings of oxidant injury to the brain might lead to profound alterations in the biophysical properties of the membrane, e.g. degree of fluidity, which in turn might greatly impair normal neuronal function.

### Example 2

Methods and Materials

CSF from 24 different subjects was collected following appropriate informed consent. Twenty-two subjects had autopsies performed in 1996 or 1997. All AD patients had been diagnosed with probable AD during life. Control subjects were age-matched individuals without clinical evidence of dementia or other neurological disease; each of these individuals had annual neuropsychological testing with all test scores in the normal range. Ventricular CSF (VF) was collected from each subject as part of a rapid autopsy protocol. Mean post mortem intervals were 2.9±0.3 hour in control subjects and 2.7±0.2 hour in AD patients; all samples were collected within 4.5 hours of death. APOE genotype was determined post mortem in all cases (Saunders et al., 1993).

Immediately following aspiration, VF was sedimented at 1000×g for 10 minutes and 1 to 2 ml were frozen at −80° C. There was no visual contamination of aspirates with blood, nor was apolipoprotein B detected in immunoblots of VF (Montine et al., 1997). Brains were evaluated using standard criteria (Khachaturian, 1985; Mirra et al., 1991). Patients with brainstem or cortical Lewy body formation, or significant cerebrovascular disease were excluded. Control subjects demonstrated only age-associated alterations. Braak staging was performed on all cases (Braak, 1991).

CSF aspirated intra vitam from the lumbar cistern (LF) was analyzed in two additional patients. Both of these patients were being evaluated for neurological disease and LF was obtained for diagnostic purposes. Both samples were free of contamination by blood and had standard clinical chemistry values within normal ranges. Ultimate diagnoses for these two patients were optic neuritis and malignant lymphoma. LF was handled and stored as described for VF.

Free F₂IsoP in 1 to 2 ml of CSF were quantified using stable isotope dilution methods employing gas chromatography/negative ion chemical ionization mass spectrometry (GC/NICIMS) as described (Morrow et al., 1990; Morrow et al., 1997). In seven patients, F₂-IsoP-like compounds were quantified that are derived from docosahexaenoic acid, the F₄-neuroprostanes (F₄-NP); these were quantified by a modification of the above GC/NICIMS method as described (Roberts et al., 1997).

Hypothesis testing for continuous data was performed with unpaired t-tests. Discontinuous data were compared with the chi-squared test. Single dimension linear regression analysis and Spearman's ranked correlation were performed using Prism 2.0 software.

Results

All 22 VF samples analyzed in this study were from subjects who participated in a rapid autopsy program. Clinical, pathological, and F₂-IsoP data for these 22 cases are presented in the Table 2. Age and gender ratios were characteristic for patients with late-onset AD and were matched to control subjects. Duration of disease was typical for the group of AD patients. Brain weight was significantly lower while Braak stage was significantly higher in AD patients compared to control subjects. APOE4 frequency in control subjects was similar to the value reported for the general population (Mahley, 1988).

Average VF F₂-IsoP levels in AD patients were significantly increased compared to control subjects (Table 2). The ranges of VF F₂-IsoP values were 12 to 68 pg/ml in control subjects and 46 to 137 pg/ml in AD patients. Single dimension linear regression analysis demonstrated a significant correlation between F₂-IsoP levels and brain weight (−0.3 pg/ml per gm, r²=0.32, P<0.01, Figure), but not with subjects' age (r²=0.06), body weight (0.04), or post mortem interval (r²=0.01). F₂-IsoP levels tended to increase with increasing duration of dementia; however, this relationship was not statistically significantly in these 11 AD patients. Ranked correlations showed that increasing F₂-IsoP levels were significantly correlated with increasing Braak stage (P<0.001), but not the number of APOE4 alleles, for all 22 subjects. When analysis was restricted to AD patients only, neither Braak stage nor the number of APOE4 alleles was significantly correlated with F₂-IsoP levels.

Recently, a series of F₂-IsoP-like compounds derived from peroxidation docosahexaenoic acid were described (Roberts et al., 1997); because docosahexaenoic acid is found primarily in the CNS, these compounds are termed F₄-neuroprostanes (F₄-NP). There was sufficient VF available for analysis of F₄-NP levels in four of the AD patients and three control subjects. Indeed, average VF F₄-NP levels were 110±12 pg/ml in these AD patients and 64±8 pg/ml in control subjects (P<0.05). VF F₂-IsoP and F₄-NP levels showed near perfect linear correlation in these seven subjects (r²=0.97, P<0.001).

In order to establish the feasibility of determining CSF F₂-IsoP levels during life, CSF aspirates were analyzed from the lumbar cistern (LF) in two additional patients with suspected neurological disease but normal CSF. LF free F₂-IsoP levels in these two patients were 30 and 32 pg/ml, approximating the VF levels in control subjects and demonstrating the potential of measuring F₂-IsoP levels during life.

Discussion

AD is associated with increased lipid peroxidation in diseased regions of brain that has been studied post mortem. While this approach has the advantage of coupling biochemical data with pathological verification of AD, two critical disadvantages have been that the assays used cannot be easily performed intra vitam and many are not entirely specific for lipid peroxidation. In the present study, free F₂-IsoP concentrations were measured, specific products of free radical-catalyzed peroxidation of arachidonic acid, in CSF from clinically and pathologically defined subjects. The results showed that average VF F₂-IsoP levels in AD patients were significantly greater than in carefully documented control subjects. Moreover, VF F₂-IsoP levels were inversely correlated with brain weight. Also, in a limited manner, the feasibility of measuring F₂-IsoPs intra vitam was demonstrated in CSF aspirates from lumbar cistern. There was no correlation between VF F₂-IsoP levels and the number of APOE4 alleles in the study; however, the number of patients was small and this lack of association with APOE genotype needs to be addressed definitively in a larger series of patients.

In the present study, F₂-IsoP levels in VF from control subjects were similar to average plasma levels in healthy human volunteers (Morrow et al., 1997), raising the possibility that free F₂-IsoP equilibrates between plasma and intrathecal compartments and suggesting that VF F₂-IsoP in control subjects is derived, at least in part, from plasma. However, several points argue that elevated VF F₂-IsoP levels in AD patients are derived from brain. First, numerous studies have consistently associated AD with regionally increased oxidative damage to brain (Markesbery, 1997), but have not consistently observed evidence of increased systemic oxidative stress (Markesbery, 1997; Ahlskog et al., 1995). Also, in the present study coincident elevations in VF F₄-NP and F₂-IsoP concentrations were demonstrated, the former being derived from docosahexaenoic acid that is extensively enriched in the CNS (Kuksis, 1978).

CSF F₂-IsoP concentration can serve as a biomarker of CNS lipid peroxidation in patients with AD. There is no other quantifiable biomarker of AD that is significantly correlated with reduced brain weight, a manifestation of cerebral atrophy, and that can be measured during life. Quantification of CSF F₂-IsoP concentration has utility as an intra vitam index of disease progression or response to therapeutic intervention.

### Example 3

Frontal lobes of brain from aged 9-month old female apoE-1-mice backbred eight generations to the C57B6/J strain and identically aged C57B6/J wild type mice were examined and determined total lipid content as well as F₂-isoP and F₄-neuroP levels. There were no differences in the tissue concentrations of phospholipid, cholesterol, triglyceride, or eight different fatty acids including AA and DHA, the substrates for isoP's and neurop's, respectively. In contrast, both F₂-isoP and F₄-neuroP tissue concentrations were significantly elevated in the same region of brain of apoE-1-mice. The concentration of DHA was three times greater than AA in apoE+/+ and apoE -1-mice. In contrast, the ratio of F₄-neuroP to F₂-isoP is 82 in apoE+/+ mice and 190 in apoE-1-mice, consistent with the in vitro observation that DHA is more vulnerable to oxidation than AA. The D+E ring forms for isoP's and neuroP's have not been measured in these mice.

| | AA | DHA | F₂-isoP | F₄-neuroP |
|---|---|---|---|---|
| | (ug/mg) | (ug/mg) | (pg/mg) | (pg/mg) |
| ApoE +/+ | 2.0 + 0.3 | 5.9 ± 0.9 | 1.7 ± 0.3 | 140.4 ± 48.3 |
| ApoE −/− | 1.9 ± 0.2 | 5.7 ± 0.8 | 2.4 ± 0.2* | 455.8 ± 122.6* |

Nine-month old mice, either apoE −/− or wild type controls, were sacrificed and one frontal lobe used to determine AA and DHA levels while the other frontal lobe was used to quantify F₂-isoP and F₄-neuroP levels. All values are means±SEM with n=4 different animals. *P≦0.01 for t-test comparing values from apoE −/− with apoE +/+ animals.

Human Subjects. Complete absence of apoE as in apoE −/− mice obviously is distinct from inheriting different apoE isoforms. Neuronal culture experiments have indicated that apoE isoforms have varying anti-oxidant activities with apoE2>apoE3>apoE4. Studies in human subjects have observed trends, although not statistically significant, toward increased levels of lipid peroxidation with inheritance of APOE4; however interpretation has been limited by the indirect and nonspecific indices used and by small sample sizes.

Ongoing experiments in the laboratory, designed to develop CSF F₂-isoP's and F₄-neuroP's as biomarkers of brain lipid peroxidation in living patients, have established the feasibility of the tissue-based studies proposed here. For these experiments, CSF was obtained post mortem from the lateral ventricles as part of a rapid autopsy protocol and was shown to be free of red blood cells or detectable apoB. Elevated F₂-IsoP levels were demonstrated in CSF of AD patients compared to carefully characterized age-matched control subjects. More importantly, CSF F₂-isoP levels are inversely correlated with brain weight (an index of brain atrophy, FIG. 8) and positively correlated with Braak stage (a histopathological index of AD severity) providing the first in vivo evidence that brain lipid peroxidation may be part of the progression of AD. In a limited number of patients, significantly increased neuroP's were demonstrated in AD patients compared to controls.

TABLE

| | | Female to | Brain weight | | Alleles as | F₂-isoP |
|---|---|---|---|---|---|---|
| | Age (yr) | Male | (g) | Braak Stage | APOE4 | (pg/ml) |
| Control | 82.2 ± 1.8 | 8:3 | 1233 ± 32 | 1.7 ± 0.4 | 12% | 46 ± 4 |
| (n = 11) | | | | | | |
| AD (n = 11) | 78.4 ± 1.6 | 7:4 | 1090 ± 51* | 5.8 ± 0.1# | 50% | 72 ± 7+ |

Value are means ± SEM, percentage of APOE4 with respect to total number of APOE alleles, or the number of male and female subjects. Unpaired t-test yielded *P = 0.05, +P = 0.01, or #P < 0.001 for control subjects vs AD patients as indicated. Ages of AD patients and control subjects were not significantly different.

### Example 4

Methods

### Preparation of Synthetic E2-IsoK [8(R)-acetyl-9-(R)-formyl-12(S)-hydroxy-5(Z), 10(E)-heptadecadienoic Acid]

The O-tert-butyl-dimethylsilyl ether, isopropylidine precursor of E2-IsoK, 8-acetyl-9-(3,3-dimethyl-2,4-dioxolanyl)-12-(t-butyldimethylsilyloxy) heptadeca-5(Z),10(E)-dienoic acid, was synthesized based on previously published methods. The precursor was then hydrolyzed in 2:1 acetic acid-water, oxidized with NaIO₄, quenched with ethylene glycol, purified, and the identity and concentration were determined by NMR as reported.

Adduction of Ovalbumin With Reactive Aldehydes

100 μM of chicken egg ovalbumin (Sigma, St. Louis, Mo.) was incubated with either vehicle (ethanol), 1 mM 4-hydroxynonenal (Cayman Chemical, Ann Arbor, Mich.) or 1 mM E₂-IsoK in 1× phosphate-buffered saline (GibcoBRL, Grand Island, N.Y.) for 4 hours at 37° C. Aliquots of individual fractions were then frozen at −70° C. until Western analysis. Samples were placed into sample buffer (Invitrogen, Carlsbad, Calif.), heated to 70° C. for 10 minutes, electrophoresed on 10% acrylamide gels (Invitrogen, Carlsbad, Calif.) and then transfered to PDVF membrane (Millipore, Belford, Mass.). The membrane was washed 2 times with Tween-Tris buffered saline (TTBS), incubated for 1 hour in the blocking solution (5% dried non-fat milk in TTBS) and for 1 hour in the presence of anti-ovalbumin monoclonal antibody OVA-14 (Sigma,St. Louis, Mo.) at 1:500. After 2 washes with TTBS, the membrane was processed further with horsadish peroxidase-conjugated sheep anti-mouse secondary antibody (Amersham, Piscataway, N.J.) at 1:7,500 dilution for 1 hour and detected by chemiluminescence (ECL; Amersham, Piscataway, N.J.).

Adduction of Aβ₁₋₄₂ With Reactive Aldehydes.

10 μM of Aβ₁₋₄₂ (a peptide comprising the first 42 residues of Aβ) (Sigma, St. Louis, Mo.) was incubated with either vehicle, 10 μM 4-HNE or 10 μM E₂-IsoK in 1× phosphate-buffered saline (GibcoBRL, Grand Island, N.Y.) for 24 hours at room temperature. Samples were electrophoresed on a 4-12% polyacrylamide gel (Invitrogen, Carlsbad, Calif.) and transferred to an Immobilon P PVDF membrane (Millipore, Belford, Mass.). The membrane was then exposed to a rabbit polyclonal anti-β-amyloid peptide primary antibody (Zymed, laboratories, Inc., San Francisco, Calif.) at 1:1,000 dilution for 1 hour. The membrane was then incubated with a donkey anti-mouse secondary antibody conjugated with horseradish peroxidase (Amersham, Piscataway, N.J.) at 1:7,500 dilution for 1 hour. Blots were detected by chemiluminescence as above.

Adduction of Tau Protein With Reactive Aldehydes: Immunoblot.

4 μM of human recombinant tau protein (Sigma, St. Louis, Mo.) was incubated with either vehicle, 4 μM 4-HNE or 4 μM E₂-IsoK in 1× phosphate-buffered saline (GibcoBRL, Grand Island, N.Y.) for 24 hours at room temperature. The samples were then electrophoresed and transferred to a PVDF membrane and the blot was further processed to a chemiluminescence detection method as described above. The primary antibody used was a rabbit anti-human tau (Dako, Carpinteria, Calif.) at 1:500 for 1 hour. The secondary antibody used was a horseradish peroxisase-linked donkey anti-mouse IgG (Amersham, Piscataway, N.J.) at 1:7,500 dilution for 1 h our.

Immunodot Blots.

4 μM of recombinant human microtubule associated protein tau, 4R isoform was incubated with 0 to 40 μM E₂-IsoK in 1× phosphate-buffered saline (GibcoBRL, Grand Island, N.Y.) for 4 hours at room temperature. Sample buffer was then added to each sample. 6.25 μg protein for each sample was loaded into duplicate wells of Dot-Blot apparatus (BioRad, Hercules, Calif.) in order to transfer onto trans-blot pure nitrocellulose membrane (BioRad, Hercules, Calif.). The membrane was then blocked using 5% dried non-fat milk and incubated with the mouse monoclonal antibody Alz50 (a kind gift from Dr Peter Davies) at 1:100 dilution. The membrane was then exposed to an anti-mouse IgM antibody (Cappel, ICN, Costa Mesa, Calif.). at 1:25,000 and paired helical filamentous-like tau was visualized using enhanced chemiluminescence.

Tissue

Brain tissues were obtained at autopsy from six patients with AD (2 males, 4 females; mean age at death 80.83±1.82 years) and six age-matched controls (4 males, 2 females; mean age at death 87.33±4.5 years). Materials were obtained from the Alzheimer's Disease Research Center at the Sanders-Brown Center on Aging, University of Kentucky. All AD patients were diagnosed as having probable AD. Controls died from non-neurological disease. Autopsies were performed within 4 hours after death and all tissue sections dissected at the autopsy were kept frozen at −80° C. until used.

NK-lysyl Lactam Adducts Purification and Analysis.

The purification and analysis of NKs-lysyl lactam adducts were performed as previously described. Briefly, tissue samples were ground in cold ethanol solution (containing 5 mg of butylated hydroxytoluene (BHT) and 50 mg of triphenylphosphine (TPP)/100 ml) (Aldrich, Milwaukee, Wis.). 0.05 ml cold ethanol solution/mg tissue was added and proteins were precipitated by centrifugation at 2000 rpm at 4° C. for 10 minutes. Proteins were then resuspended in 3 ml of cold MeOH (containing BHT and TPP) and 3 ml of 0.4 N KOH (containing Trolox) and hydrolyzed under argon for 2 hours at 37° C. Proteins were then reprecipitated in cold ethanol (0.05 ml/mg tissue, containing BHT and TPP) and subsequently with 0.05 ml cold Folch solution/mg tissue, and washed with 0.05 ml cold methanol/mg tissue (containing BHT and TPP). Proteins were resuspended in 1× phosphate-buffered saline and heated at 95° C. for 10 minutes. After cooling, pronase (Calbiochem, La Jolla, Calif.) was added (1 g/g of starting tissue weight) and incubated overnight at 37° C. The digest was then heated at 95° C. for 10 minutes to inactivate the pronase and after cooling, aminopeptidase M (Calbiochem, La Jolla, Calif.) was added (400 μl/g of starting tissue weight) and incubated 18 hours at 37° C. The digest was applied to a 1 g Oasis cartridge (Waters Associates, Milford, Mass.), filtered and purified by HPLC using a gradient consisting of 20 mM ammonium acetate with 0.1% acetic acid to 5 mM ammonium acetate /MeOH/acetic acid (10:90:0.1, v/v/v) on a 4.6×250 mm Macrosphere 300 C18 column (MacMod Analytical, Chadds Ford, Pa.). HPLC fractions containing radioactivity from NK-lysyl adduct internal standard were combined, re-extracted with an Oasis cartridge and analyzed by liquid chromatography/electrospray ionization/tandem mass spectrometry (LC/ESI/MS/MS). Internal standards were synthesized as follow. 25 mg of docosahexaenoic acid (Nu-Chek-Prep, Inc., Elysian, Minn.) was oxidized in presence of [¹³C₆]-lysine (2 mg) (Cambridge Isotope Laboratories, Inc., Andover, Mass.) and [³H]-lysine (50×10⁶ cpm) (NEN, Boston, Mass.). Adducts were extracted with Oasis cartridge and HPLC as describe above. One ml fractions were collected and aliquots containing radioactivity were analyzed and quantified by LC/ESI/MS/MS using selective reaction monitoring of the fragmentation of the [MH]⁺ ion to a specific daughter ion for the lactam adduct.

Statistical Analysis.

Data are presented as mean±SEM. SPPS 9.01 was used for statistical analysis. Analysis of variance (ANOVA) was performed to evaluate the statistical significance of difference between groups with the Bonferroni correction. Statistical significance was assigned to the level of p<0.05.

Results

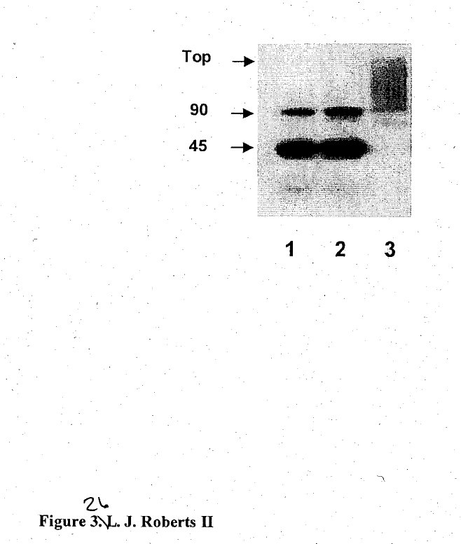

The ability of 4-HNE and a synthetic IsoK to cross-link proteins using ovalbumin as a model were compared. A synthetic isomer [8(R)-acetyl-9(R)-formyl-12(S)-hydroxy-5(Z), 10(E)-heptadecadienoic acid] (E2-IsoK), which has a hydrophobicity and a reactivity similar to that off other IsoK and NK isomers generated by the IsoP and NP pathways were used. As demonstrated by SDS-PAGE, incubation of ovalbumin (which contains 20 lysine residues) with 10 molar equivalents of E₂-IsoK for 4 hours at 37° C. resulted in the disappearance of the 45 kDa band and the appearance of a broad smear of higher molecular weight bands, consistent with crosslinked species (FIG. 26). Unlike E₂-IsoK, incubation of ovalbumin with 10 molar equivalent of 4-HNE for 4 hours failed to produce any evidence of crosslinked species.

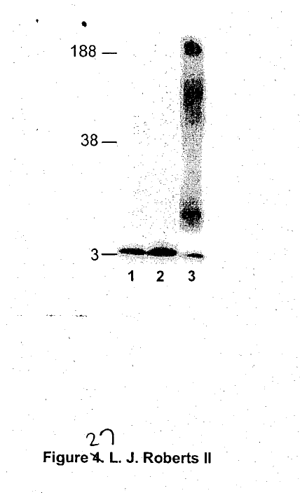

Senile plaques and neurofibrillary tangles are two hallmarks of Alzheimer's disease. The major protein component of the core of the senile plaques is Aβ. Aβ₁₋₄₀ and Aβ₁₋₄₂ are the two principal forms of Aβ, 60% of all plaques containing Aβ₁₋₄₂ while 31% contain Aβ₁₋₄₀. Aβ₁₋₄₂ is also more fibrillogenic in vitro than Aβ₁₋₄₀. 4-HNE has been shown to cause progressive covalent crosslinking of Aβ₁₋₄₀ in a dose-dependent manner but very high concentrations are required and extensive cross-linked species were not observed. Therefore, it was thought to be of interest to compare the effect of E₂-IsoK and 4-HNE on Aβ₁₋₄₂. 10 μM of Aβ₁₋₄₂ (which contains 2 lysine residues) was incubated for 24 hours at room temperature with 1 molar equivalent E2-IsoK or 4-HNE. Western blot analysis using the anti-β-amyloid peptide antibody revealed a near complete loss of the 3.5 kDa band and an appearance of high molecular weight bands consistent with crosslinking of Aβ₁₋₄₂ (FIG. 27). In contrast, under the same conditions, there was no evidence of cross-linking following incubation of 10 μM of Aβ₁₋₄₂ with 1 molar equivalent of 4-HNE.

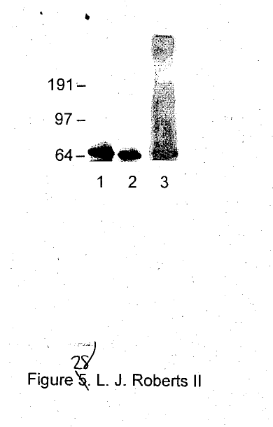

Neurofibrillary tangles seen in AD are bundles of paired helical filaments, which are comprised largely of tau protein. The cause of the tau aggregation is currently not understood, but crosslinking of tau by lipid peroxidation products has been proposed as a mechanism. It has been shown that 4-HNE cross-links tau into high molecular weight species following incubation of high concentrations of 4-HNE (1 mM) with P19 neuroglial cells. The ability of E₂-IsoK and 4-HNE to cross-link human recombinant tau in vitro was compared. 4 μM of tau was incubated with 4 mM of E₂-IsoK for 24 hours at room temperature. Western blot analysis was then performed using an anti-tau antibody. The results revealed the disappearance of the tau 65 kDa band with the appearance of new high molecular weight bands (FIG. 28). By contrast, no apparent oligomerization occurred following incubation of tau with 4-HNE under the same conditions.

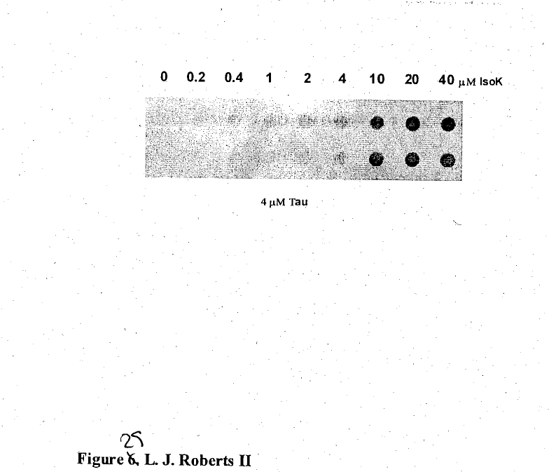

The monoclonal antibody Alz50 recognizes specific conformational epitopes on paired helical filament-tau in AD brain and can be used to assess to the presence of neurofibrillary tangles in post-mortem Alzheimer's disease brain. Reaction of crosslink-forming aldehydes, such as 4-HNE, with tau can generate the Alz50 epitope in vitro. However, again very high concentrations of 4-HNE (mM) are required for this effect and 4-HNE is only capable of generating the Alz50 epitope on phosphorylated tau although phosphorylation is not an absolute requirement for generation of the epitope. Since the formation of the Alz50 epitope requires cross-linking of two lysine residues, the underlying mechanism of cross-linking by IsoKs and NKs, it was reasoned that adduction of tau with E₂-IsoK generates the Alz50 epitope. Recombinant 4 μM of human tau was adducted with E₂-IsoK (0.2 to 40 μM) and then determined Alz50 binding by dot-blot analysis (FIG. 29). E₂-IsoK induces the Alz50 epitope and the creation of the Alz50 epitope by reaction with E₂-IsoK occurs beginning at low micromolar concentrations and increases further in a concentration-dependent manner. Notably, the creation of the Alz50 epitope by E₂-IsoK occurred in non-phosphorylated tau.

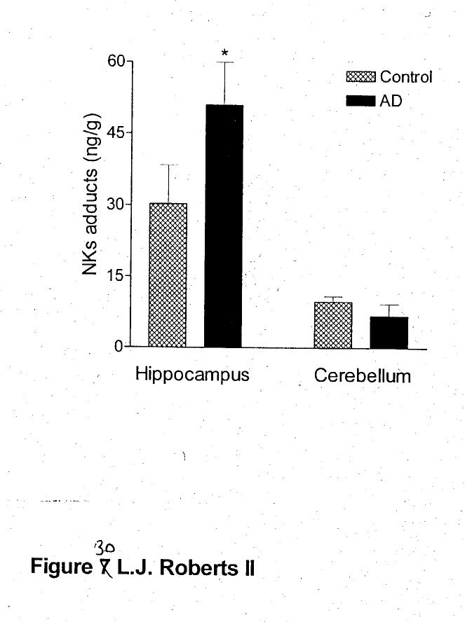

Because DHA is the major fatty acid in neuronal membranes and is more susceptible than AA to oxidation, NKs play a more prominent role as neurotoxins than IsoKs in settings of oxidant injury to the brain. Moreover, it was previously demonstrated that NKs protein adducts can be detected in normal human brain. Therefore, whether NK adducts are increased in Alzheimer's disease brain was investigated. NKs lactam adducts were quantified after enzymatic digestion of proteins to individual amino acids in hippocampus and cerebellum from AD patients and controls (FIG. 30). Levels of NK-lysyl adducts were found to be significantly higher in the hippocampus, a disease-affected area of brain in AD (44.04±5.87 ng/g tissue) than in controls (27.06±2.68 ng/g tissue) (p<0.05). In contrast, in non disease-affected area of brain, cerebellum, levels of NKs adducts in AD brain (10.87±1.94 ng/g tissue) were no different from controls (14.69±2.74 ng/g tissue).

Discussion

Alzheimer's disease is pathologically characterized by the formation of amyloid-positive senile plaques and intraneuronal tau-positive neurofibrillary tangles and is associated with selective neuronal death. The mechanism(s) responsible for tau and Aβ aggregation in the AD brain as well as for neuronal loss are still poorly understood. There is accumulating evidence supporting a role for oxidative stress in the pathogenesis of AD. Products of both the IsoP and NP pathways of lipid peroxidation are increased in AD but NPs are increased to a greater extent in the brain of AD patients. Because NKs are highly reactive products of the NP pathway and because they exhibit a unique proclivity to cross-link proteins, whether NKs are involved in the pathogenesis of AD was explored. Because a number or studies have suggested that 4-HNE may be involved in the pathogenesis of AD, the effects of a synthetic IsoK derived from the IsoP pathway on protein cross-linking/aggregation with that of 4-HNE was compared.

The synthetic IsoK covalently modified and extensively cross-linked ovalbumin under conditions in which 4-HNE failed to induce any appreciable cross-linking. These comparative studies were extended to a peptide and protein more relevant to AD, Aβ₁₋₄₂, and human recombinant tau. IsoK induced extensive cross-linking of both Aβ₁₋₄₂ and tau under conditions in which 4-HNE again failed to induce any appreciable cross-linking.

E2-IsoK induced the Alz50 epitope in tau. The monoclonal antibody Alz50 recognizes a specific epitope on tau protein in fibrillar pathology in AD brain. It has been shown that experimental agents that can cross-link two lysine residues are capable of inducing the Alz50 epitope on tau. The ability of the IsoK to induce the Alz50 epitope is therefore understandable in that IsoKs and NKs induce protein cross-linking between lysine residues. Although 4-HNE has been shown capable of inducing the Alz50 epitope, high concentrations (mM) are required and there is an obligatory requirement that tau be phosphorylated for this effect. In striking contrast, it was found that the IsoK induced the Alz50 epitope in non-phosphorylated tau at low micromolar concentrations.

The above findings show that NKs is one of the most attractive candidates in regards to products of lipid peroxidation that can be involved in protein cross-linking and aggregation in AD. Other recent findings from the laboratory further support this notion. Impairment of proteasome function is also a feature of AD and, interestingly, significant reduction of activity only is observed in the same brain regions in which increased oxidative damage has occurred. Moreover, inhibition of proteasome function in neuronal cultured cells and neurons in vivo has been shown to induce apoptosis. IsoK adducted proteins are very poorly degraded by the proteasome. In addition, IsoK adducted proteins potently inhibit the proteasome from degrading normal substrates. Both of these effects are expected to lead to protein accumulation and aggregation and promote apoptosis. In this regard, it was also found that incubation of E2-IsoK with P19 neuroglial cells was associated with cytotoxicity and inhibition of proteasome function at submicromolar concentrations approximately 100-fold lower than concentrations of 4-HNE required to induce cytotoxicity in these cells.

Critical to the hypothesis that NKs play a role in protein aggregation and cross-linking in AD is demonstrating that levels of NK protein adducts are in fact increased in the brain of patients with AD. Toward this goal applicants were able to show that NK protein adducts are in fact significantly increased in AD. Importantly, these increases were present in a disease affected area of brain, the hippocampus, but levels in a area of brain unaffected by the disease, the cerebellum, were no different from controls.

In summary, oxidative stress has been strongly implicated in the pathogenesis of AD and products of lipid peroxidation, specifically 4-HNE, have been suggested to be involved in protein aggregation and cross-linking in AD. In the studies reported herein, the potential that a recently discovered new class of highly reactive products of lipid peroxidation, NKs are involved in the protein aggregation and cross-linking in AD was explored. Using a synthetic IsoK to model the effects of NKs, it was found that NKs are capable of inducing extensive protein cross-linking of Aβ₁₋₄₂ and tau under conditions in which no cross-linking was observed for 4-HNE. Moreover, incubation of IsoK with tau created the Alz50 epitope at concentrations that are more than two orders of magnitude lower than observed with HNE and did not require phosphorylation of tau. Finally, and importantly, levels of NK adducted proteins were significantly and selectively increased in disease affected area of brain from patients with AD. These studies identify NKs as a highly attractive candidate for being involved in protein aggregation and cross-linking and neurodegeneration in AD.

### Example 5

Isoprostanes (IsoPs) are prostaglandin (PG)-like compounds that are generated in vivo, nonenzymatically, as products of free radical-induced peroxidation of arachidonoyl lipids. Their formation proceeds via PGH2-like bicyclic endoperoxide inter-mediates, which are reduced to form F-ring IsoPs (F2-IsoPs) or undergo rearrangement to form D-ring and E-ring IsoPs and isothromboxanes. Recently, it was reported that IsoP en-doperoxide intermediates also undergo rearrangement to form highly reactive γ-ketoaldehyde levuglandin-like compounds. These nonenzymatically generated γ-ketoaldehydes are termed isoketals (IsoKs) to distinguish them from levuglandins formed by rearrangement of the cyclooxygenase endoperoxide intermediate, PGH2. These extremely reactive molecules form covalent adducts with lysine residues on proteins at a rate that exceeds that of 4-hydroxy-2-nonenal (4-HNE) by orders of magnitude, which is considered to be one of the most reactive aldehydes generated as a product of lipid peroxidation. Moreover, they exhibit a remarkable proclivity to cross-link proteins. It was previously shown that IsoKs initially form a reversible Schiff base adduct, which then proceeds through a pyrrole to stable lactam and hydroxylactam adducts.

Docosahexaenoic acid (DHA) (22:6ω3) is a polyunsaturated fatty acid uniquely enriched in the brain and retina, especially in synaptic membranes and in photoreceptor cells. Astrocytes play an important role in the delivery of DHA to the blood-brain barrier endothelial cells and to neurons. Although the physiologic basis for why DHA is enriched in the brain and retina remains unclear, reduced levels of DHA are associated with disturbances in visual acuity, behavior, and learning in young animals and humans. Because DHA is highly concentrated in nervous system tissue, these compounds are termed neuroprostanes (NPs). Analogous to the formation of IsoPs, the formation of NPs also proceeds through bicyclic endoperoxide intermediates that not only are reduced to F-ring compounds but also undergo rearrangement in vivo to form D- and E-ring NPs. Therefore, the hypothesis that IsoK-like compounds could also be generated as rearrangement products of the NP pathway was explored. The interest in the possibility that NKs can be formed derives from the fact that free radicals have been implicated in the pathogenesis of a wide variety of neurodegenerative disorders, including Huntington's disease, amyotrophic lateral sclerosis, Parkinson's disease, and Alzheimer's disease. Furthermore, reactive aldehydes are thought to be key mediators of oxidant injury because of their capacity to covalently modify proteins and DNA. Thus, generation of NKs can induce neuronal injury due to their reactivity and can be involved in the formation of protein crosslinks, a common feature in neurodegenerative diseases. The notion that NKs, if formed, participate in the pathogenesis of Alzheimer's disease is strengthened by the previous finding of Applicants that F₄-NP levels are significantly increased in cerebrospinal fluid from patients with this disease.

The pathway by which NKs can be generated is shown in FIGS. 1, A-C. Five docosahexaenoyl radicals are initially formed that are then converted to eight peroxyl radicals following the addition of oxygen. These undergo endocyclization followed by further addition of molecular oxygen to form eight bicyclic endoperoxide intermediate regioisomers, which can then rearrange to form eight D₄-NK and eight E₄-NK regioisomers. The designation “D” and “E” is a carryover from the established prostaglandin nomenclature for PGD and PGE and levuglandins E and D to indicate the location of the keto group. Each regioisomer is theoretically comprised of eight racemic diastereoisomers for a total of 128 D₄-type and 128 E₄-type NKs. In accordance with the nomenclature system for IsoPs that has been approved by the Eicosanoid Nomenclature Committee, the eight regioisomers are designated by the carbon number on the side chain of the precursor endoperoxide intermediates where the hydroxyl group was located, with the carboxyl carbon as C1.

Experimental Procedures

Materials

Docosahexaenoic acid was purchased from Nu-Chek-Prep, Inc. (Elysian, Minn.). Undecane, N-N-dimethylformamide, ammonium acetate, Trolox, and triphenylphosphine were from Aldrich; pentafluorobenzyl bromide, methoxyamine HCL, sodium borohydride, and butylated hydroxytoluene were from Sigma; Pronase and porcine aminopeptidase M (60 units/ml) were from Calbiochem; C18 Sep Pak cartridges and Oasis cartridges were from Waters Associates (Milford, Mass.); and [¹³C₆]L-lysine and [²H₃]methoxyamine HCl were from Cambridge Isotope Laboratories, Inc. (Andover, Mass.). L-[4,5⁻³H]Lysine was from PerkinElmer Life Sciences; N,O-bis(trimethylsilyl)trifluoro-acetamine was from Regis Chemical (Morton Grove, Ill.); N,O-[²H₉]bis(trimethylsilyl)trifluoroacetamine was from CDP isotopes (Pointe-Claire, Quebec, Canada). 4.6×250-mm Macrosphere 300 C18 column and 2.1×15-mm XD8-C8 column were from MacMod Analytical (Chadds Ford, Pa.). Male Harlan Sprague-Dawley rats were from Harlan Sprague-Dawley, Inc. (Indianapolis, Ind.).

Oxidation of DHA

5 mg of DHA were oxidized in vitro in 1× phosphate- buffered saline using an iron/ADP/ascorbate mixture (1 mM/200 mM/100 mM) for 2 hours as described.

Purification and Analysis of NKs by Gas Chromatography (GC)/Negative Ion Chemical Ionization (NICI)/Mass Spectrometry (MS)

The purification and analysis of NKs followed similar procedures used for purification and analysis of IsoKs. Following oxidation of DHA, compounds were converted to O-methyloxime derivatives by treatment with a 3% aqueous solution of methoxyamine HC1. The pH of the reaction mix was then adjusted to 3, and the samples were extracted using a C18 Sep Pak cartridge. The compounds were then converted to a pentafluorobenzyl ester derivative, purified by TLC using the solvent heptane/ethyl acetate (60:40, v/v), converted to a trimethylsilyl ether derivative, and quantified by GC/NICI/MS using [²H₄]PGE₂ as an internal standard and a modification of the method used to purify and analyze IsoKs. The region extending from 1.5 cm above to 2.5 cm above an O-methyloxime pentafluorobenzyl ester derivative of [²H₄]PGE2 standard was scraped. This area was determined to contain NKs by analyzing sequential small cuts of the TLC plates. NKs were detected by GC/NICI/MS employing selected ion monitoring for the M-CH₂C₆F₅ ions (m/z 505 for NKs and m/z 528 for the [²H₄]PGE₂ internal standard). Catalytic hydrogenation was performed as described previously.

Purification and Analysis of F₄-NPs and IsoKs by GC/NICI/MS

Purification and analysis of F₄-NPs and IsoKs by GC/NICI/MS was performed as described.

Formation and Analysis of NK-lysyl Adducts

10 mg of DHA was oxidized as described above in the presence of 10 mg of lysine. To reduce and stabilize Schiff base adducts, 1/10 volume-of 1 M sodium borohydride in DMF was added and allowed to incubate for 30 minutes at 4° C. The sample was extracted with an Oasis cartridge and analyzed by liquid chromatography (LC)/electrospray ionization (ESI)/MS/MS in the positive ion mode as described. The auxiliary gas pressure was 10 p.s.i., and the sheath gas pressure was 70 p.s.i. The voltage on the capillary was 20 V, and the tube lens voltage was 80 V. The capillary temperature was 200° C. Collision-induced dissociation (CID) of molecular ions of putative NK-lysyl adducts was performed from −20 eV to −40 eV with 2.6-millitorr collision gas, scanning daughter ions between 50 and 550.

Preparation and Oxidation of Rat Brain Synaptosomes

Synaptosomes were prepared from brain of Harlan Sprague-Dawley rats according to the method of Janowsky et al. Lipid peroxidation was initiated by the addition of an iron/ADP/ascorbate mixture as described above. Incubations were carried out at 37° C. for 4 hours, and the samples were then placed at −80° C. to terminate the reactions.

Analysis of NK-lysyl Adducts in Rat Brain Synaptosomes

Following oxidation, 1 volume of 0.4 N KOH (containing 3 mM Trolox) was added for base hydrolysis, and the mixture was incubated under argon for 2 hours at 37° C. After neutralization of the sample with 5 N HCl, 10 volumes of cold ethanol (containing 5 mg of butylated hydroxytoluene (BHT) and 50 mg of triphenylphosphine (TPP)/100 ml) were added, and the proteins were precipitated by centrifugation at 2000 rpm at 4° C. for 10 minutes. Proteins were then reprecipitated in 10 volumes of cold Foich solution and washed with 10 volumes of MeOH (each containing BHT and TPP). Proteins were resuspended in 1× phosphate-buffered saline and heated to 98° C. for 5 minutes. After cooling, Pronase (3 mg/mg of starting protein weight) was added, and the mixture was incubated overnight at 37° C. Samples were then heated at 98° C. for 5 minutes to inactivate the Pronase, and after cooling, aminopeptidase M (1 μl/mg starting protein weight) was added, and the digest was incubated at 37° C. for 18 hours. The digest was extracted with an Oasis cartridge as described above and purified by HPLC using a 4.6×250 mm Macro-sphere 300 C18 column. The solvent system employed was a gradient consisting of 20 mM ammonium acetate with 0.1% acetic acid (solvent A) to 5 mM ammonium acetate/MeOH/acetic acid (10:90:0.1, v/v/v) (solvent B). The flow rate was 1 ml/min beginning at 100% A, followed by an increase to 40% B over 5 minutes and then to 100% B over 14 minutes. The column was then washed with 100% B for 10 more minutes. HPLC fractions containing radioactivity from NK-lysyl adduct internal standards were combined, reextracted with Oasis cartridges, and analyzed by LC/ESI/MS/MS as described above. Internal standards were formed by oxidation of 25 mg of DHA in the presence of [¹³C₆]lysine (2 mg) and [³H]lysine (50×10⁶ cpm). Adducts were extracted with Oasis cartridge and HPLC as described above. Fractions were collected every min and aliquots containing radioactivity were analyzed by LC/ESI/MS/MS. HPLC fractions containing NK-lysyl adducts were combined, and the concentration was calculated from the specific activity of the [³H]lysine.

Analysis of NK-lysyl Adducts in Human Brain

Human cerebral cortices were ground in cold Folch solution (containing BHT and TPP). Proteins were precipitated and resuspended in 3 ml of cold MeOH (containing BHT and TPP) and 3 ml of 0.4 N KOH (containing Trolox) for the base hydrolysis. Proteins were then precipitated, washed, and subjected to complete enzymatic digestion to individual amino acids. Adducts were then extracted by Oasis cartridge and HPLC (using the same solvents as above and a flow rate of 1 ml/min beginning at 100% A followed by an increase to 40% B over 14 minutes and then to 100% B over 16 minutes) and analyzed by LC/ESI/MS/MS as described above.

Results

Evidence of the Formation of NKs During Oxidation of DHA In Vitro

Previously, there was shown that oxidation of arachidonic acid (AA) in vitro results in the formation of IsoKs. Thus, we initially explored whether NKs are also formed in vitro during oxidation of DHA with iron/ADP/ascorbate. A representative selected current chromatogram obtained from this analysis is shown in FIG. 2. The two peaks in the lower m/z 528 chromatogram represent the syn- and anti-O-methyloxime isomers of the internal standard [²H₄ ]PGE₂. The predicted M-CH₂C₆F₅ ion for the pentafluorobenzyl ester, O-methyl-oxime, trimethylsilyl ether derivative NKs is m/z 505. In the upper ion current chromatogram are a series of m/z 505 peaks eluting at a longer retention time compared with PGE₂. These peaks are consistent with NKs, which are expected to have a longer GC retention time than PGE2 because of their two additional carbon atoms.

Additional analyses further supported the identity of these compounds as NKs. No peaks were seen in the m/z 504 ion current chromatogram, indicating that the peaks in the m/z 505 chromatogram are not natural isotope peaks of compounds generating an ion of less than m/z 505. Analysis of the putative NKs as a [³H₉] trimethylsilyl ether derivative resulted in a shift in the 505 peaks eluting after the arrow in FIG. 2 upwards to m/z514, indicating the presence of one hydroxyl group. When analyzed as a [²H₃]-O-methyloxime derivative, the m/z 505 peaks eluting after the arrow shifted upwards 6 Da to m/z 511, indicating the presence of two carbonyl groups. Analysis following catalytic hydrogenation is shown in FIG. 3. Prior to catalytic hydrogenation, there were no peaks present 8 Da above m/z 505 at m/z 513. However, following hydrogenation, intense new peaks appear at m/z 513 with a concomitant loss of the peaks in the m/z 505 ion current chromatogram. This indicated that the compounds contained four double bonds. Collectively, these data indicate that the compounds represented by the chromatographic peaks eluting after, but not before, the arrow in the m/z 505 ion current chromatogram have the type and number of functional groups and double bonds predicted for NKs.

Relative amounts of NKs and F₄-NPs formed during oxidation of DHA were compared. The amounts of NKs formed are less than the amounts of F₄-NPs (FIG. 4A). The amount of NKs generated exceeded the amount of IsoKs formed during co-oxidation of equal molar amounts of DHA and AA with iron/ADP/ascorbate by 3.1-fold (FIG. 4B).

Evidence for the Formation of NK-lysyl Adducts

It was previously demonstrated that IsoKs covalently adduct to lysine residues with remarkable rapidity (within seconds). IsoKs initially form an unstable reversible Schiff base adduct, which then proceeds through a pyrrole to stable lactam and hydroxylactam adducts (FIG. 5). To determine whether NKs form covalent adducts with lysine in vitro, DHA was oxidized with iron/ADP/ascorbate in the presence of lysine. Adducts were then analyzed, after reduction by sodium borohydride, by LC/ESI/MS. Selected ion current chromatograms monitoring m/z 491 and 503 from these analyses are shown in FIG. 6. The predicted [MH]⁺ ion for the dehydrated reduced Schiff base NK lysine adduct is m/z 491. This is consistent with the previous observation that IsoKs not only undergo reduction during treatment with the sodium borohydride but also dehydration. The predicted [MH]⁺ ions for the NK lysine lactam adducts is m/z 503. The presence of multiple m/z 491 and 503 peaks is consistent with the formation of multiple NK-lysyl adduct isomers (see FIG. 1).

To further substantiate the structural identity of these compounds as Schiff base and lactam adducts, the compounds were analyzed by LC/ESI/MS/MS. CID of the putative dehydrated reduced Schiff base adducts produced daughter ions at m/z 473 and 346 (FIG. 7A). CID of the putative NK-lysyl lactam adducts produced relevant daughter ions at m/z 485, m/z 467, m/z 356, m/z 338, and m/z 84 (FIG. 7B). The ions at m/z 473 in the Schiff base CID spectrum and the ions at m/z 485 and m/z 467 in the lactam CID spectrum represent the loss of one molecule of H₂O (m/z 473, 485) and two molecules of H₂O (m/z 467). Other daughter ions present in these CID spectra can be assigned the structures shown in FIG. 8 based on analogous ions present in the CID spectra of IsoK-lysyl adducts.

Formation of NK-lactam Protein Adducts in Rat Brain Syn-aptosomes

Synaptosomes (composed of sealed off neuronal and glial processes) are a widely used model for the study of central nervous system gray matter metabolism. The formation of NK-lactam adducts in nonoxidized synaptosomes and in synaptosomes following oxidation for 4 hours with iron/ADP/ascorbate were compared. NK-lysyl lactam adducts were isolated, after complete enzymatic digestion of proteins to individual amino acids, and quantified following base hydrolysis. Adducts were analyzed by LC/ESI/MS/MS utilizing selected reaction monitoring of the transition of the [MH]⁺ ions for the synaptosomal lactam adducts (m/z 503) and NK [¹³C₆]lysine lactam internal standards (m/z 509) to the specific respective CID ions m/z 84 and 89. The internal standards were obtained by oxidation of DHA in the presence of [¹³C₆]lysine and [³H]lysine. The lactam adducts were detected in nonoxidized synaptosomes at a level of 0.09 ng/mg of protein (FIG. 9A), and levels increased 19-fold to 1.71 ng/mg of protein following oxidation (FIG. 9B). The pattern of peaks representing synaptosomal lactam adducts differs somewhat from the pattern obtained for the internal standard. This can be explained by the observation that there appears to be a steric influence of phospholipids on the formation of different isomers from esterified substrate. The pattern of peaks representing lactam adducts in nonoxidized synaptosomes also differs somewhat from the patterns detected in oxidized synaptosomes. This is due to variation in absolute recovery of the NK adduct isomers in the pooled HPLC fractions collected due to the large number of isomers that elute over a broad range, as was also seen for peaks representing the internal standards.

Detection of NK-lysyl Adducts in Vivo in Human Brain

Proteins from frozen human cerebral cortex were precipitated, delipidated, and treated with or without base hydrolysis before complete proteolysis. Levels of NK-lysyl lactam adducts in the cerebral cortex were 9.9±3.7 ng/g of brain tissue (η=4) (FIG. 10). The amount of adducts detected was not different from the levels measured when proteins had not been subjected to base hydrolysis, indicating that NK-lactam adducts were not esterified to phospholipids. This is consistent with the observation that IsoK-lactam protein adducts are not associated with phospholipids.

Discussion

These studies have identified a novel class of IsoK-like compounds that are formed by free radical-induced peroxidation of DHA both in vitro and in vivo. The motivation for exploring whether IsoK-like compounds are formed via the NP pathway stems from the fact that DHA is uniquely enriched in neural and retinal tissues; DHA comprises about one-third and 30-65% of total fatty acids in aminophospholipids of gray matter and rod outer segments, respectively.

Oxidative damage has been strongly implicated in the pathogenesis of a number of neurological disorders. The brain is especially sensitive to oxidative injury because of its high content of polyunsaturated fatty acids, its high oxygen consumption rate, and its relative paucity of antioxidant defenses compared with other tissues. In this regard, NPs and IsoPs are readily detectable in normal brain tissue, suggesting a level of ongoing oxidant stress in the brain. It was of interest to find that NK-lysyl protein adducts are also readily detectable in normal brain tissue, suggesting that proteins are being covalently modified by NKs even in the normal state. At present, it is not known if NPs exert biological activity. However, because of their capacity to covalently modify proteins, adduction of key proteins by NKs can be highly injurious to neurons. This may take on particular relevance in pathologic disorders involving oxidant injury. This notion is supported by the findings that levels of NPs and/or IsoPs are significantly increased, indicative of enhanced oxidant injury, in both Huntington's disease and Alzheimer's disease. Reactive aldehydes derived from lipid peroxidation have been suggested to play a key role in the pathogenesis of neurodegenerative processes. The reactive aldehydes most intensively studied have been 4-HNE and 4-hydroxy-2-hexenal, formed from oxidation of AA and DHA, respectively, and malondialdehyde. Protein-bound 4-HNE levels are increased in Alzheimer's disease entricular fluid, pyramidal neuron cytoplasm, and neurofibrillary tangles in Alzheimer's disease brain and in Parkinson's disease nigral neurons. Modification of proteins by 4-HNE impairs the function of neuronal glucose transporter GLUT-3 and the astrocytic glutamate transporter GLT-1 and causes disruption of neuronal microtubules. Although 4-HNE is considered to be one of the most cytotoxicly reactive aldehydes formed from lipid peroxidation, it is of interest and particular relevance that it was previously shown that IsoKs adduct to lysine residues at a rate that exceeds that of 4-HNE by several orders of magnitude. Relevant to the hypothesis that NKs can be important effector molecules in the pathobiology of oxidative neuronal injury are the data obtained from the 2,5-hexanedione, the reactive metabolite that is responsible for the neurotoxicity of η-hexane. 2,5-Hexanedione is a γ-diketone that reacts with the ε-amine group of lysine with reaction chemistry similar to that of NKs. γ-Diketone neuropathy is 30 characterized by cross-linking of neurofilaments, via the formation of pyrrole adducts, leading to axonal atrophy and swelling. Thus, the neurotoxic effects of NKs would be expected to be similar to that of 2,5-hexanedione.

It is interesting to note that the amounts of NKs generated during co-oxidation of equivalent amounts of DHA and AA in vitro were greater than the amounts of IsoKs formed. This is consistent with the fact that DHA is more susceptible than AA to oxidation. This is also in accord with the findings that (a) NP levels are higher than levels of IsoPs in normal human brain, (b) levels of NPs are increased in brain from patients with Alzheimer's disease, whereas IsoPs are not, and (c) levels of NPs are higher than levels of IsoPs in cerebrospinal fluid from both control subjects and patients with Alzheimer's disease. Collectively, this suggests that NKs formed by the NP pathway plays a more prominent role as neurotoxins in settings of oxidant injury to the brain than IsoKs formed by the IsoP.

In summary, these studies have elucidated the formation of highly reactive γ-ketoaldehydes NKs as products of the NP pathway of free radical-induced peroxidation of DHA, both in vitro and in vivo. This identifies a new class of molecules are involved in the formation of protein adducts and protein cross-links in neurodegenerative diseases, a common feature of these disorders, and contribute to the injurious effects of other oxidative pathologies in the brain.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

TABLE I
Levels of F₂-IsoPs and F₄-NPs measured esterified to lipids in whole
normal rat brain (n = 4) and in brain cortex
from newborn pig (n - 3)
F₂-IsoPs and F₄-NPs were measured as free compounds following
base hydrolysis of a Folch lipid extract of brain tissue as described
under “Experimental Procedures.” The data are expressed as nano-
grams of F₂-IsoPs and F₄-NPs measured per g of wet weight of tissue.

| | | | p value (F₂-IsoP vs |
|---|---|---|---|
| | F₂-IsoPs | F₄-NPs | F₄-NP |
| | ng/g | | |
| Whole brain from ± | 10.3 ± 3.1 | 7.0 ± 1.4 | >0.05 |
| normal rat | | | |
| Brain cortex from | 2.9 ± 0.4 | 13.1 ± 0.8 | <0.0001 |
| newborn pig | | | |

TABLE 2
Clinical, Pathological, and F₂-IsoP Data for Subjects
With Post Mortem Examination

| | | | Duration | | | | |
|---|---|---|---|---|---|---|---|
| | | | Of | | | % alleles | |
| | Age | Female | Disease | Brain | Braak | As | F₂-IsoP |
| | (yr) | to Male | (yr) | Weight (g) | Stage | APOE4 | (pg/ml) |
| Control | 82.2 ± 1.8 | 8:3 | 0.0 | 1233 ± 32 | 1.7 ± 0.4 | 12% | 46 ± 4 |
| (n = 11) | | | | | | | |
| AD | 78.4 ± 1.6 | 7:4 | 7.2 ± 1.2 | 1090 ± 51* | 5.8 ± 0.1# | 50%\hat{ } | 72 ± ^{z}+ |

### References

Ahiskog J, Utti R, Low P, Tyce G, Nickander K, Petersen R, and Kokmen E. No evidence for systemic oxidant stress in Parkinson's or Alzheimer's disease. Movement Disorders 1995; 10:566-73.

Atsmon, J., Sweetman, B. J., Baertschi, S. W., Harris, T. M., and Roberts, L. J. (1990) Biochemistry29, 3760-3765.

Attallah, A., Payakkapan, W., Lee, J., Carr, A., and Brazelton, E. (1974) Prostaglandins 5, 69-71.

Bernoud-Hubac, N., Davies, S S., Boutaud, O., Montine, T J., Roberts II, L. J. Formation of Highly Reactive γ-Ketoaldehydes (Neuroketals) as Products of the Neuroprostane Pathway, The Journal of Biological Chemistry, Vol. 276, No. 33, Issue of Aug. 17, 2001, pp. 30964-90670.

Bernoud-Hubac, N., Davies, S S., Amarnath, V., Markesbery, W R., Montine, T J., Roberts II, L. J. Enhanced Covalent Modification of Protiens by Neuroketals in Alzheimer's Disease Brain.

Bourre, J. M., Pascal, G., Durand, G., Masson, M., Dumont, O., and Piciotti, J. (1984) U. Neurochem. 43, 342-348.

Boyland, E., and Chasseaud, L. F. (1968) Biochem, J. 109, 651-661.

Braak H and Braak E. Neuropathological standing of Alzheimer-related changes. Acta Neuropathol 1991; 82-239-59.

Bui, T., and Straus, D. S. (1998) Biochem. Biophys. Acta. 1397, 31-42.

Conner, W. E., Neuringer, M., and Reisbick, S. (1992) Nutri Rev. 50, 21-29.

Dratz, E. A., and Deese, A. J. (1986) Health Effects of Polyunsaturated Fatty Acids in Seafoods (Simopoulos, A. P., Kifer, R. R., and Martin, R. E., eds) pp. 319-351, Academic Press, Orlando, Fla.

Forman, B. M., Tontonoz, P., Chen, J., Brun, R. P., Spiegelman, B. M., and Evans, R. M. (1995) Cell83, 803-812.

Fukushima, J. (1992) Prostaglandins Leukotrienes Essent. Fatty Acids 47, 1-12.

Fukushima, M. (1990) Eicosanoids 3, 189-199.

Hirata, Y., Hayashi, H., Ito, S., Kikawa, Y., Ishibashi, M., Sudo, M., Miyazaki, H., Fukushima, M., Narumiya, S., and Hayaishi, O (1988) J. Biol. Chem. 263,16619-16625.

Honn, K. V., and Marnett, L. J. (1985) Biochem. Biophys. Res. Commun. 129, 34-40.

Jonsson, H. T., Middleditch, B. S., Schexnayder, M. A., and Desiderio, D. M. (1976) J. Lipid. Res. 17, 1-6.

Khachaturian, Z. S. Diagnosis of Alzheimer's disease. (1985) Arch. Neurol 42, 1097-1105.

Kim, I.-K., Lee, J.-H., Sohn, H.-W., Kim, H.-S., and Kim, S.-H. (1993) FEBS Lett. 321, 209-214.

Kliewer, S. A., Lenhard, J. M., Willson, T. M., Patel, I., Morris, D. C., and Lehmann, J. M. (1995) Cell 83, 813-819.

Knight, J. A. (1997) Ann. Clin. Lab. Sci. 27, 11-25.

Kuksis A. Fatty acid composition of glycerolipids of animal tissues. Handbook of Lipid Research. Edited by A. Kuksis. New York, Plenum Press, 1978, p. 381-442.

Longmire, A. W., Swift, L. L., Roberts, L. J., II, Awad, J. A., Burk, R. F., and Morrow, J. D. (1994) Biochem. Pharmacol. 47,1173-1177.

Lovell, M., Ehmann, W., Mattson, M., and Markesbery, W. Elevated 4-hydroxynonenal in ventricular fluid in Alzheimer's disease. (1997) Neurobiol. Aging 18, 457-461.

Mahley R W. Apolipoprotein E: cholesterol transport protein with expanding role in cell biology. Science 1988; 240:622-630.

Markesbery, W. R. (1997) Free Radic. Biol. Med. 23, 134-147.

Markesbery, W. R. Oxidative stress hypothesis in Alzheimer's disease. Free Radic Biol Med 1997; 23:134-147.

Melegos, D. M., Diamandis, E. P., Oda, H., Urade, Y., and Hayaishi, O. (1996) Clin. Chem. 42, 1984-1991.

Middledtich, B. S., (1975) Prostaglandins 9, 409-411.

Mirra, S. S., Heyman, A., McKeel, D., Sumi, S. M., Crain, B. J., Brownlee, L. M., Vogel, F. S., Hughes, J. P., van Belle, G., and Berg, L. The Consortium to Establish a Registry for Alzheimer's Disease (CERAD). Part II. Standardization of the neuropathologic assessment of Alzheimer's disease. (1991) Neurology 41, 479-486.

Montine, T. J., Montine, K. S., and Swift, L. L. Central nervous system lipoproteins in Alzheimer's disease (1997) Am. J. Pathol. 151, 1571-1575.

Moore, S. A., Yoder, E., Murphy, S., Dutton, G. R., and Spector, A. A. (1991) J. Neurochem. 56, 518-524.

Morrow, J. D., and Roberts, L. J., II (1994) Methods Enzymol. 233, 163-174.

Morrow, J. D., and Roberts, L. J., II The isprostanes: unique bioactive products of lipic peroxidation. (1997) Prog. Lipid Res. 36, 1-21.

Morrow, J. D., Awad, J. A., Boss, H. J., Blair, I. A., and Roberts, L. J., II (1992) Proc. Natl. Acad. Sci. U.S.A. 89,10721-10725.

Morrow, J. D., Awad, J. A., Wu, A., Zackert, W. E., Daniel, V. C., and Roberts, L. J., II (1996) J. Biol. Chem. 271, 23185-23190.

Morrow, J. D., Harris, T. M., and Roberts, L. J., II (1990) Anal. Biochem. 184, 1-10.

Morrow, J. D., Hill, K. E., Burk, R. F., Nammour, T. M., Badr, K. F., and Roberts, L. J., II A series of prostaglandin-like compounds produced in vivo in humans by a non-cyclooxygenase, free radical catalized mechanism. (1990) Proc. Natl. Acad. Sci. U.S.A. 87, 9383-9387.

Morrow, J. D., Minton, T. A., Mukundan, C. R., Campbell, M. D., Zackert, W. E., Daniel, V. C., Badr, K. F., Blair, I. A., and Roberts, L. J., II (1994) J. Biol. Chem. 269, 4317-4326.

Pace-Acsiak, C. R. and Wolfe, L. S. (1971) J. Chromatogr. 56,129-135.

Morrow, J. D., Prakash, C., Awad, J. A., Duckworth, T A., Zakert, W. E., Blair, I. A., Oates, J. A., and Roberts, L. J., II (1991) Anal. Biolchem 193, 142-148

Narumiya, S., Ohno, K., Fujiwara, M., and Fukushima, M. (1986) J. Pharmacol. Exp. Ther. 239, 506-511.

Narumiya, S., Ohno, K., Fukushima, M., and Fujiwara, M. (1987) J. Pharmacol. Exp. Ther. 242, 306-311.

Pace-Asciak, C. R. (1989) Adv. Prostaglandin Thromboxane Leukotriene Res. 18, 322.

Ricote, M., Li, A. C., Willson, T. M., Kelly, C. J., and Glass, C. K. (1998) Nature 391, 79-82.

Roberts, L. J., II, and Morrow, J. D. (1997) Biochim. Biophys. Acta. 1345,121-135.

Roberts L and Morrow J. Formation of novel isoprostane-like compounds for docosahexaenoic acid. R A Isma, R A Armstrong, R W Kelly, and R Wilson, Editors. Fourth International Congress on Essential Fatty Acids and Eicosanoids 1997; AOCS Press, Champaigne, Ill. In press.

Salem, N., Jr., and Niebylske, C. D. (1995) Mol. Membr. Biol. 12, 131-134.

Salem, N., Jr., Kim, H.-Y., and Yergery, J. A. (1986) Health Effects of Polyunsaturated FattyAcids in Seafoods (Simopoulos, A. P., Kifer, R. R., and Martin, R. E., eds) pp. 263-317, Academic Press, Orlando, Fla.

Santoro, M. G. (1997) Trends Microbiol. 5, 276-281.

Saunders, A M, Strittmatter W J, Schmechel D, St. George-Hyslop P H, M. A P-V, Joo S H, Rosi B L, Gusella J F, Crapper M D, Alberts M J, Hulette C, Crain B, Goldgaber D, and Roses A D. Association of apolipoprotein E allele epsilon 4 with late-onset familial and sporadic Alzheimer's disease. Neurology 1993; 43(8):1467-72.

Shahabi, N. A., Chegini, N., and Wittliff, J. L. (1987) Exp. Cell Biol. 55, 18-27.

Simonian, N. A., and Coyle, J. T. (1996) Annu. Rev. Pharmacol. Toxicol. 36, 83-106.

Skinner, E. R., Watt, C., Besson, J. A. O., and Best, P. V. (1993) Brain 116, 717-725.

Strittmatter W J and Roses A D. Apolipoprotein E and Alzheimer's disease. Proc Natl Acad Sci 1995; 92:4725-4727.

Taber, D. F., Morrow, J. D., and Roberts, L. J., II (1997) Prostaglandins 53, 63-67.

Tokugawa, Y., Kunishige, I., Kubota, Y., Shimoya, K., Nobunaga, T., Kimura, T., Saji, F., Murata, Y., Eguchi, N., Oda, H., Urade, Y., and Hayaishi, O. (1998) Biol Reprod. 58, 600-607

What is claimed is:

1. A method to assess oxidative stress in vivo comprising:
(a) measuring the amount of neuroprostanes and metabolites thereof in a biological sample before the ex vivo development of neuroprostanes in the sample;
(b) comparing the measured amount of the neuroprostanes and metabolites with a control; and
(c) assessing oxidative stress in vivo based on the comparison in step b.

2. The method according to claim 1, further including the step of storing the biological sample prior to said measuring step.

3. The method according to claim 2, wherein the stored sample is maintained at −70° C.

4. The method according to claim 1, wherein the sample is cerebrospinal fluid.

5. A marker for oxidative stress comprising neuroprostanes, isothromboxane-like compounds and isolevuglandin-like compounds derived from DHA, and metabolites thereof, which increase in a biological sample compared to a control sample during oxidative stress.

6. The marker according to claim 5, wherein said marker is F₂-neuroprostane.

7. The marker according to claim 5, wherein said marker is E₂-neuroprostane.

8. The marker according to claim 5, wherein said marker is D₂-neuroprostane.

9. The marker according to claim 5, wherein said marker is an isothromboxane-like compound.

10. The marker according to claim 5, wherein said marker is an isolevuglandin-like compound.

11. The marker according to claim 5, wherein said marker is a neuroketal.

12. A diagnostic tool for determining the presence of a neurodegenerative disease comprising neuroprostane, isothromboxanes and metabolites thereof, which are increased in a biological sample compared to a control sample.

13. The diagnostic tool according to claim 12, wherein said neurodegenerative disease is Alzheimer's disease.

14. A metabolite of neuroprostanes isothromboxane-like compounds, and isolevuglandin-like compounds formed by one process from the group consisting essentially of beta oxidation, omega oxidation double bond reduction, dehydrogenation of the side chain hydroxyl groups, and reduction of the ring carbonyl to a hydroxyl group.
